(19) United States
(12) Patent Application Publication
(10) Pub. No.: US 2013/0338021 A1
(43) Pub. Date: Dec. 19, 2013

(54) METHODS AND COMPOSITIONS FOR THE DETECTION OF COMPLICATIONS OF DIABETES

(71) Applicant: WISCONSIN ALUMNI RESEARCH FOUNDATION, Madison, WI (US)

(72) Inventors: Melanie Laura Dart, Madison, WI (US); Hans Sollinger, Madison, WI (US); Debra A. Hullett, Oregon, WI (US)

(73) Assignee: Wisconsin Alumni Research Foundation, Madison, WI (US)

(21) Appl. No.: 13/838,428

(22) Filed: Mar. 15, 2013

Related U.S. Application Data
(60) Provisional application No. 61/659,548, filed on Jun. 14, 2012.

(51) Int. Cl. G01N 33/50

(52) U.S. Cl. CPC G01N 33/5091
USPC 506/9; 435/29; 435/7.92; 435/7.1

(57) ABSTRACT

The present disclosure provides methods and compositions for determining the presence of or predisposition to insulin resistance, diabetes, and complications of diabetes in a subject. The methods relate to measuring the capacity of a subject's peripheral blood mononuclear cells (PBMCs) to induce physiological and/or morphological changes characteristic of fibrosis in cultured.

### CROSS-REFERENCE TO RELATED APPLICATIONS

This application claims the benefit of U.S. Application No. 61/659,584, filed Jun. 14, 2012, which is incorporated herein by reference in its entirety.

### STATEMENT OF GOVERNMENT SUPPORT

This invention was made with government support under AI066219 and DK077354 awarded by the National Institutes of Health. The government has certain rights in the invention.

### FIELD OF TECHNOLOGY

The present technology relates to methods and compositions for determining the presence of or predisposition to insulin resistance, diabetes and complications of diabetes in a subject.

### BACKGROUND

Currently over 346 million people worldwide have diabetes; in the U.S., 26 million people have diabetes with close to an additional 80 million people being pre-diabetic. Diabetes is the 7^{th} leading cause of death and yet, is likely to be underreported. The annual medical costs associated with diagnosed diabetes is upwards of $175 billion in the US alone, and estimated at $375 billion globally. The frequency of diabetes is ever increasing, particularly type 2 diabetes, which is why the CDC has referred to this disease as the “epidemic of our time.”

Type 2 diabetes is the type of diabetes that occurs later in life and its development is preceded by the development of insulin resistance, which often occurs years before diabetes is diagnosed. Insulin resistance is a condition in which the cells of the body become resistant to the hormone, insulin. During the initial stages of insulin resistance development the patient remains normoglycaemic, despite the beginnings of insulin resistance of the cells, because the pancreas is able to produce enough insulin to overcome this resistance. As the resistance in cells continues to increase, eventually the pancreas can no longer produce enough insulin, whether due to high demand or loss of beta cell function, leading to impaired glucose tolerance and diabetes. In addition to diabetes development, insulin resistance has also been associated with fatty liver, arteriosclerosis, skin tags, and reproductive abnormalities in women.

In general practice, diagnosis of type 2 diabetes relies on measuring glucose levels in conjunction with fasting insulin levels. However, this only gives the physician an indication as to whether insulin resistance is present or not in patients without diabetes. A firm diagnosis cannot be made simply based on this, since the lab techniques for measuring insulin can vary, and there is no absolute value that meets a definition. Generally, a level above the upper quartile in the fasting state in someone without diagnosed diabetes is considered to be abnormal. Typically, a diagnosis of type 2 diabetes is given after a detailed patient history, patient physical examination, and other risk factors are determined. There are additional confirmative tests for insulin resistance, such as the euglycemic insulin clamp or intravenous tolerance testing, however these tests are expensive and complicated, not lending them to broad patient screening programs.

The present disclosure provides methods for diagnosis and prognosis of diabetic nephropathy, insulin resistance, and type 2 diabetes.

### SUMMARY

In one aspect, the present disclosure provides a method for identifying a subject as having a predisposition to diabetic nephropathy, comprising: (a) co-culturing a biological sample from the subject in vitro with one or more renal cell lines; (b) maintaining the co-culture for a sufficient time for the biological sample to induce physiological changes in the renal cells; and (c) detecting the physiological changes in the renal cells; wherein the subject is asymptomatic for diabetic nephropathy.

In some embodiments, the biological sample comprises peripheral blood mononuclear cells (PBMCs) or urine. In some embodiments, the subject is an individual diagnosed as having, suspected of having, or predisposed to having one or more of type 1 diabetes, type 2 diabetes, insulin resistance, normalbuminuria, or microalbuminuria.

In some embodiments, a sufficient time comprises about 12 to about 72 hours. In some embodiments, a sufficient time comprises about 24 hours.

In some embodiments, the physiological changes comprise changes in cell or cell culture morphology. In some embodiments, the changes in cell or cell culture morphology comprise changes associated with fibrosis. In some embodiments, the changes in cell or cell culture morphology associated with fibrosis comprise one or more of spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, enhanced individual cell migration. In some embodiments, the changes in cell or cell culture morphology comprise changes in cell area, compactness, eccentricity, extent, solidity, angle between neighbors, radial distribution, angular second movement, contrast, difference entropy, difference variance, entropy, inverse difference moment, sum average, sum variance, or variance.

In some embodiments, the physiological changes comprise changes in protein levels. In some embodiments, the changes in protein level comprise an increase in one or more of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII, IL-1β, IL10, MIP-1α, MIP-1β, phospho-CREB, DPPIV, EGF, EGFR, TIM-1, TNF-α, VEGF, annexin V, angiotensin, CXCL16, MCP-1, GRO-α, or IL-1Ra. In some embodiments, the changes in protein level comprise a decrease in one or more of phospho-HSP27, JAM-C, podocalyxin, and VAP-1.

In some embodiments, the detecting comprises microscopy, immunostaining, ELISA, protein arrays, western blotting or flow cytometry.

In one aspect, the present disclosure provides a method for identifying a subject as diabetic, comprising: (a) co-culturing a biological sample from the subject in vitro with one or more renal cell lines; (b) maintaining the co-culture for a sufficient time for the biological sample to induce physiological changes in the renal cells; and (c) detecting the physiological changes in the renal cells; wherein the subject is asymptomatic for diabetic nephropathy.

In some embodiments, the biological sample comprises peripheral blood mononuclear cells (PBMCs) or urine. In some embodiments, the subject is an individual suspected of having or predisposed to having one or more of type 1 diabetes, type 2 diabetes, insulin resistance, normalbuminuria, microalbuminuria, or macroalbuminuria.

In some embodiments, a sufficient time comprises about 12 to about 72 hours. In some embodiments, a sufficient time comprises about 24 hours.

In some embodiments, the physiological changes comprise changes in cell or cell culture morphology. In some embodiments, the changes in cell or cell culture morphology comprise changes associated with fibrosis. In some embodiments, the changes in cell or cell culture morphology associated with fibrosis comprise one or more of spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, enhanced individual cell migration. In some embodiments, the changes in cell or cell culture morphology comprise changes in cell area, compactness, eccentricity, extent, solidity, angle between neighbors, radial distribution, angular second movement, contrast, difference entropy, difference variance, entropy, inverse difference moment, sum average, sum variance, or variance.

In some embodiments, the physiological changes comprise changes in protein levels. In some embodiments, the changes in protein level comprise an increase in one or more of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII, IL-1β, IL10, MIP-1α, MIP-1β, phospho-CREB, DPPIV, EGF, EGFR, TIM-1, TNF-α, VEGF, annexin V, angiotensin, CXCL16, MCP-1, GRO-α, or IL-1Ra. In some embodiments, the changes in protein level comprise a decrease in one or more of phospho-HSP27, JAM-C, podocalyxin, and VAP-1.

In some embodiments, the detecting comprises microscopy, immunostaining, ELISA, protein arrays, western blotting or flow cytometry.

In one aspect, the present disclosure provides a method for identifying agents or compounds with the capacity to regulate physiological and/or morphological changes characteristic of fibrosis, comprising: (a) co-culturing a biological sample from a subject in vitro with one or more renal cell lines; (b) maintaining the co-culture for a sufficient time for the biological sample to induce physiological changes in the renal cells; (c) contacting the renal cells with a candidate agent; (d) detecting physiological changes in the renal cells; and (e) comparing the physiological changes to those of a control sample.

In some embodiments, the biological sample comprises peripheral blood mononuclear cells (PBMCs) or urine. In some embodiments, the subject is an individual diagnosed as having, type 1 diabetes, type 2 diabetes, insulin resistance, normalbuminuria, microalbuminuria, or macroalbuminuria.

In some embodiments, a sufficient time comprises about 12 to about 72 hours. In some embodiments, a sufficient time comprises about 24 hours.

In some embodiments, the physiological changes comprise changes in cell or cell culture morphology. In some embodiments, the changes in cell or cell culture morphology comprise changes associated with fibrosis. In some embodiments, the changes in cell or cell culture morphology associated with fibrosis comprise one or more of spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, enhanced individual cell migration. In some embodiments, the changes in cell or cell culture morphology comprise changes in cell area, compactness, eccentricity, extent, solidity, angle between neighbors, radial distribution, angular second movement, contrast, difference entropy, difference variance, entropy, inverse difference moment, sum average, sum variance, or variance.

In some embodiments, the physiological changes comprise changes in protein levels. In some embodiments, the changes in protein level comprise an increase in one or more of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII, IL-1β, IL10, MIP-1α, MIP-1β, phospho-CREB, DPPIV, EGF, EGFR, TIM-1, TNF-α, VEGF, annexin V, angiotensin, CXCL16, MCP-1, GRO-α, or IL-1Ra. In some embodiments, the changes in protein level comprise a decrease in one or more of phospho-HSP27, JAM-C, podocalyxin, and VAP-1.

In some embodiments, the detecting comprises microscopy, immunostaining, ELISA, protein arrays, western blotting or flow cytometry. In some embodiments, the renal cells are contacted with the agent prior to, simultaneous to, or subsequent to culturing in conjunction with the biological sample.

In one aspect, the present disclosure provides a kit for identifying a subject as diabetic or as having a predisposition to diabetic nephropathy, comprising: (a) a compilation of biomarkers predictive of the presence of diabetes or predisposition to diabetic nephropathy; (b) one or more positive and/or negative control biological samples; (c) optionally a compilation of morphological changes predictive of the presence of diabetes or predisposition to diabetic nephropathy; (d) optionally a vessel for the co-culture the biological sample with a renal cell line; and (e) instructions for use.

In some embodiments, the compilation of physiological changes predictive of the presence of or predisposition to diabetic nephropathy and/or insulin resistance comprises a compilation of parameters describing cell or cell culture morphology.

In some embodiments, the parameters describing cell or cell culture morphology comprise parameters relating to one or more of spindle formation, cell elongation, cell contractility/mobility, proliferation, apoptosis, necrosis, viability, cell-cell contact, filapodial stress fibers, cytoskeletal reorganization, tight intercellular junctions, focal adhesions, individual cell migration.

In some embodiments, the parameters describing cell or cell culture morphology comprise parameters relating to one or more of cell area, compactness, eccentricity, extent, solidity, angle between neighbors, radial distribution, angular second movement, contrast, difference entropy, difference variance, entropy, inverse difference moment, sum average, sum variance, or variance.

In some embodiments, the compilation of physiological changes predictive of the presence of or predisposition to diabetic nephropathy and/or insulin resistance comprises a compilation of predictive biomarkers.

In some embodiments, the compilation of predictive biomarkers comprises one or more of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII, IL-1β, IL10, MIP-1α, MIP-1β, phospho-CREB, DPPIV, EGF, EGFR, TIM-1, TNF-α, VEGF, annexin V, angiotensin, CXCL16, MCP-1, GRO-α, IL-1Ra, phospho-HSP27, JAM-C, podocalyxin, or VAP-1.

In one aspect, the present disclosure provides a method for identifying a subject as having a predisposition to diabetes, comprising: (a) co-culturing a biological sample from the subject in vitro with one or more renal cell lines; (b) maintaining the co-culture for a sufficient time for the biological sample to induce physiological changes in the renal cells; and (c) detecting the physiological changes in the renal cells.

### BRIEF DESCRIPTION OF THE FIGURES

This application file contains at least one drawing executed in color. Copies of this patent or patent application publication with color drawing(s) will be provided by the Office upon request and payment of the necessary fee.

The drawing figures included herein depict embodiments of the present innovation. The drawings are presented by way of example, not by way of limitation.

FIG. 1 is a schematic diagram of an illustrative embodiment of the fibrosis assay of the present disclosure.

FIG. 2A-H show HK-2 cells after co-culture with peripheral blood mononuclear cells (PBMCs). Results for a healthy controls and subjects with type 2 diabetes in conjunction with diabetic nephropathy, normoalbuminuria (short-term normoA; long-term normoA), or microalbuminuria (microA), compared to positive (TGF-β-positive) and negative (untreated) controls are shown in panels A-I. Diabetic nephropathy comprises macroalbuminuria, stage 1V diabetic kidney disease, or end stage renal failure. Panels A-D show unstained HK-2 cells, and panels E-H show immunofluorescence of HK-2 cells after co-culture with PBMCs from healthy and type 2 diabetics with nephropathy, compared to positive (“TGF-β-positive”) and negative controls (“untreated”). Cells were stained for F-actin (green), ZO-1 (red) and DNA (blue). Panel I shows fibrosis ratios for healthy subjects, type 2 diabetics with short-term duration of disease and normoA, type 2 diabetics with long-term duration of disease and normoA, type 2 diabetics with microA, and type 2 diabetics with nephropathy. Fibrosis ratios falling above the dashed line shown in panel C are considered positive fibrosis ratios, and values falling below the dashed line are considered negative fibrosis ratios. Asterisks indicate a significant p-value when compared to healthy controls (p<0.0001) and to long-term type 2 diabetics with normoA (p<0.0001).

FIG. 3A-F shows a computational analysis of morphological changes in HK-2 cells induced by co-culture with PBMCs from a healthy subject and PBMCs from type 2 diabetics with nephropathy. Quantitative analysis of the morphological changes is given in Table 1.

FIG. 4A-C shows immunofluorescence of A549 cells after co-culture with PBMCs from a subject with chronic obstructive pulmonary disease and PBMCs from a subject with type 2 diabetics with nephropathy, compared to untreated control cells. Cells were stained for F actin (green), ZO-1 (red), and DNA (blue).

FIG. 5 shows HK-2 cells following co-culture with PBMCs from a healthy subject and a type 1 diabetic with nephropathy. Panels A-D show unstained cells (panels A, B) and immunofluorescence stained (panels C, D) HK-2 cells following co-culture with subjects' PBMCs. Cells are stained for ZO-1 (red) and DNA (blue) (panels C, D). Panel E shows fibrosis ratios for healthy subjects, type 1 diabetics with normoalbuminuria, and a type 1 diabetic with nephropathy.

FIG. 6 shows HK-2 cells following co-culture with PBMCs from a healthy subject (panels A, C, E, G, I) and a type 2 diabetic with nephropathy (panels B, D, F, H, J). Cells are stained for vimentin (panels A, B, red), collagen V (panels C, D, green), aSMA (panels E, F, green), CTGF (panels G, H, green), collagen I (panels I, J, green), and DNA (all panels, blue).

FIG. 7 shows results of quantitative immunoblotting of HK-2 cell lysates after co-culture with PBMC from healthy subjects (panels B-G, solid black bars) and type 2 diabetics with nephropathy (panels B-G, hatched bars). Lysates were analyzed by western blot for levels of vimentin, fibronectin, connective tissue growth factor (CTGF), collagen type IV (col IV), alpha smooth muscle actin (aSMA), and collagen type I (col I). The immunoblot is shown in panel A. Quantification of the immunoblot is shown in panels B-G.

FIG. 8A-F show HK-2 cells following co-culture or trans-well culture with PBMCs from a healthy subject and a type 2 diabetics with nephropathy compared to a TGF-13 positive control. Cells are stained for F-actin (green), ZO-1 (red) and DNA (blue). Fibrosis ratios are shown in panel G. The asterisks indicate a significant decrease in fibrosis ration when compared to diabetic nephropathy direct cell contact (p=0.0017).

FIG. 9 shows phosphorylated MAP kinase levels in lysates of HK-2 cells co-cultured with PBMCs from type 2 diabetics with nephropathy and type 2 diabetics with long-term disease duration but having normoalbuminuria. Panel A shows the results of an antibody array assay of HK-2 cell lysates following co-culture with subjects' PBMCs. Integers refer to the bars shown in Part C. Panel B is a map of the protein array showing the identities and positions of protein-specific antibodies on the array. Panel C shows the quantification of proteins indicated by integers in Part A as given by mean pixel density.

FIG. 10 shows the quantification of phosphorylated CREB, GSK-3α/β, JNK2, and p38-delta in HK-2 lysates following co-culture with PBMCs from subjects with type 2 diabetics with short-term disease duration and normoalbuminuria whose fibrosis assays were either positive or negative, as defined for FIG. 1 above.

FIG. 11A-C shows levels of phosphorylated JNK, p38, and ERK in HK-2 lysates following co-culture with PBMCs from healthy controls and type 2 diabetics with nephropathy as measured by ELISA.

FIG. 12 shows levels of soluble hematopoietic receptors in HK-2 cell culture supernatants following direct and trans-well culture with PBMCs from a healthy subject and type 2 diabetics with nephropathy. Panel A shows the results of antibody array assays. Integers in panel A refer to bars shown in panel C. Panel B is a map of the protein array showing the identities and positions of protein-specific antibodies on the array. Panel C shows the quantification of proteins indicated by integers in panel A as measured by mean pixel density.

FIG. 13 shows levels of kidney biomarker proteins in HK-2 cell lysates following co-culture with PBMCs from a healthy subject and type 2 diabetics with nephropathy. Panel A shows results of antibody array assays. Integers in panel A refer to bars shown in panel C. Panel B is a map of the protein array showing the identities and positions of protein-specific antibodies on the array. Panel C shows the quantification of proteins indicated by integers in panel A as measured by mean pixel density.

FIG. 14 shows illustrative levels of kidney biomarker proteins in HK-2 cell culture supernatants following co-culture with PBMCs from a healthy subject and three type 2 diabetic subjects. Panels A, C, E, and G show results of antibody array assays. Panels B, D, F, and H are maps of the protein arrays showing the identities and positions of protein specific antibodies on the arrays. Color coding indicates a relative level of protein from very little (light green) to high expression (dark red).

FIG. 15 shows levels of kidney biomarker proteins in urine samples from a healthy subject and three type 2 diabetics with nephropathy. Panel A shows results of antibody array assays. Panel B is a map of the protein array showing the identities and positions of protein-specific antibodies on the array. Panel C shows the quantification of protein levels in the healthy subject and the average values for the three type 2 diabetics with nephropathy.

FIG. 16 shows levels of non-hematopoietic soluble receptors in HK-2 cell culture supernatants following co-culture with PBMCs from a healthy subject and a type 2 diabetic with nephropathy. Panel A shows results of antibody array assays. Integers refer to bars shown in panel C. Panel B is a map of the protein array showing the identities and positions of protein-specific antibodies on the array. Panel C shows the quantification of proteins indicated by integers in panel A as measured by mean pixel density.

FIG. 17 shows levels of common analyte soluble receptors in HK-2 cell culture supernatants following co-culture with PBMCs from a healthy subject and a type 2 diabetic with nephropathy. Panel A shows illustrative results of antibody array assays. Integers refer to bars shown in panel C. Panel B is a map of the protein array showing the identities and positions of protein-specific antibodies on the array. Panel C shows the quantification of proteins indicated by integers in panel A as measured by mean pixel density.

FIG. 18 shows levels of kidney biomarkers in HK-2 cell culture supernatants following co-culture with PBMCs from a healthy subject and a pre-diabetic subject. Panel A shows illustrative results of antibody array assays. Integers refer to bars shown in panel C. Panel B is a map of the protein array showing the identities and positions of protein-specific antibodies on the array. Panel C shows the quantification of proteins indicated by integers in panel A as measured by mean pixel density.

### DETAILED DESCRIPTION

General—

The present disclosure provides methods for detecting a presence of or predisposition to diabetic nephropathy and/or insulin resistance in a subject.

The techniques and procedures described herein are generally performed according to conventional methods in the art and various general references, which are provided throughout this document. See generally, Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively. Generally, the nomenclature used herein and the laboratory procedures in cell culture, molecular genetics, organic chemistry, analytical chemistry and nucleic acid chemistry and hybridization described below are those well known and commonly employed in the art. Standard techniques are used for nucleic acid and peptide synthesis. Standard techniques, or modifications thereof, are used for chemical syntheses and chemical analyses. All references cited herein are incorporated herein by reference in their entireties and for all purposes to the same extent as if each individual publication, patent, or patent application was specifically and individually incorporated by reference in its entirety for all purposes.

### DEFINITIONS

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term that are not clear to persons of ordinary skill in the art, given the context in which it is used, “about” will mean up to plus or minus 10% of the particular term.

As used herein, “subject” refers to a mammal. In some embodiments, a subject is a human. In some embodiments a subject refers to a mammal for whom it is desired to detect a presence of or predisposition to diabetes, complications of diabetes, diabetic nephropathy, or insulin resistance. In some embodiments, the mammal is a human.

As used herein, “predisposition” to diabetic nephropathy or insulin resistance refers to the likelihood that a subject not having these conditions will develop these conditions at some point in the future. As known in the art, the predisposition of a subject to a given condition is determined by making comparison of one or more aspects of the subject's physiological characteristics to those of one or more control subjects.

As used herein, “healthy control” refers to a subject with no known fibrotic disorder or any other disease or condition.

As used herein, “co-culture” refers to the process of culturing a disease-targeted organ resident cell line (e.g., renal cell line for diabetic nephropathy; lung epithelial cell for lung chronic obstructive pulmonary disease, etc.) in vitro in direct contact with a biological sample from a subject.

As used herein, “organ resident” cell refers to a cell that is derived from the organ type that is compromised in a diseased individual. Accordingly, what cell type constitutes an organ resident cell will vary across subjects depending on what disease conditions are relevant to the subject. For example, a renal cell is an organ resident cell for an individual with nephropathy, while a lung epithelial cell is an organ resident cell for an individual with lung disease. By “renal cell line” is meant a primary or immortalized cell line derived from human or non-human kidney cells, including those derived from embryonic and non-embryonic tissues. Illustrative renal cell lines include but are not limited to HK-2 cells. In some embodiments, the renal cell line is derived from human renal proximal tubular epithelial cells, human renal epithelial cell culture model, human renal epithelial cells, or human renal cortical epithelial cells. Cell lines may be cultured according to methods known in the art, including illustrative culture conditions described herein. One skilled in the art will understand that culture conditions may be optimized according to the particular organ resident cell or cell type in use and operator preferences. In some embodiments, organ resident cells (e.g. renal cells) are cultured in direct contact with a subject's biological sample (i.e. co-culture). In some embodiments, the organ resident cells (e.g. renal cells) are cultured adjacent to the subject's biological sample, separated from the subject's biological sample by a semipermeable membrane (i.e. trans-well culture).

By “biological sample” is meant any fluid, cell, tissue, or organ derived from the subject. Illustrative biological samples include but are not limited to cells, tissues, blood, serum, plasma, saliva, urine, cerebrospinal fluid, and interstitial fluid. In some embodiments, the biological sample is peripheral blood mononuclear cells (PBMCs). In some embodiments, the biological sample is urine.

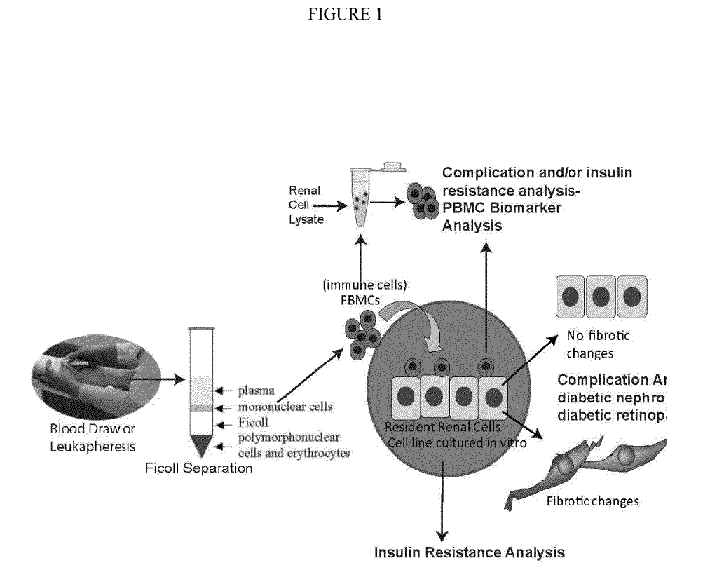

As used herein, “co-culture fibrosis assay” refers to methods exemplified by the below examples and shown in FIG. 1. According to the methods, a biological sample from a subject is co-cultured with an organ resident cell (e.g., a cell line) for a period of time sufficient for the biological sample to induce physiological changes in the organ resident cells that are characteristic of fibrosis. In some embodiments, the organ resident cells (e.g., the cell line) is a renal cell line. In some embodiments the physiological changes are morphological changes in the cell line. In some embodiments the physiological changes are increased or decreased protein expression found in the cell line, the PBMC, and/or the supernatant after co-culture. In some embodiments, the methods comprise co-culture of organ resident cells (e.g. renal cells) with a subject's biological sample. In some embodiments, the methods comprise trans-well culture of organ resident cells (e.g. renal cells) with a subject's biological sample.

As used herein, “fibrosis” refers to the formation of excess fibrous connective tissue within an organ or tissue. As used herein, physiological and/or morphological changes “characteristic of fibrosis” refers to physiological and/or morphological changes in cultured cells that typically occur during the development of fibrosis or are typically observed in fibrotic tissue. Any suitable characteristic of fibrosis may be detected using the present methods. Illustrative characteristics of fibrosis include, but are not limited to, increased spindle formation, cell elongation, increased cell contractility/mobility, increased proliferation, increased apoptosis, increased necrosis, decreased viability, reduced cell-cell contact, increased filapodial stress fibers, cytoskeletal re-organization, decreased tight intercellular junctions, formation of focal adhesions, enhanced individual cell migration; increased levels of one or more proteins selected from alpha smooth muscle actin (αSMA), angiotensin, annexin V, ANPEP, CD10, CD170, CD44H, CD59, CD99, chitinase 3-like 1, collagen I, collagen IV, complement, connective tissue growth factor (CTGF), CXCL16, Cyr61, DDR2, DPPIV, EGF, EGFR, elastin, endothelin, E-selectin, FGF, fibronectin, FSP1, galectin 1, GRO-α, GSK-3α/β, HSP47, IL10, IL15Ra, IL-17, IL-1R, IL-1Ra, IL-10, IL-2, IL-6, IL-9, IMAM-1, integrin β1, integrin β2, LFA-3, lipocalin-2, L-selectin, MCP-1, MIP-1α, MIP-1β, MMP-9 myeloperoxidase, MMPs, N-cadherin, phopho-MKK6, phospho-Akt 2, phospho-CREB, phospho-ERK, phospho-JNK2, phospho-p38δ, phosphor-RSK2, resistin, SCF, Slug, Snail, Tamps, target of rapamycin, TGF-β, TIM-1, TLR, TNF-R1, TNF-RII, TNFRSF5, TNF-α, total phospho-Akt, TRACAP, Twist; VCAM-1, VEGF, or vimentin, increased levels of an mRNA that encodes a protein selected from alpha smooth muscle actin (αSMA), angiotensin, annexin V, ANPEP, CD10, CD170, CD44H, CD59, CD99, chitinase 3-like 1, collagen I, collagen IV, complement, connective tissue growth factor (CTGF), Cyr61, DDR2, DPPIV, EGF, EGFR, elastin, endothelin, E-selectin, FGF, fibronectin, FSP1, galectin 1, GRO-α, GSK-3α/β, HSP47, IL10, IL15Ra, IL-17, IL-1R, IL-1Ra, IL-113, IL-2, IL-6, IL-9, IMAM-1, integrin β1, integrin β2, LFA-3, lipocalin-2, L-selectin, MIP-1α, MIP-β3, MMP-9 myeloperoxidase, MMPs, N-cadherin, phopho-MKK6, phospho-Akt 2, phospho-CREB, phospho-ERK, phospho-JNK2, phospho-p38δ, phosphor-RSK2, resistin, SCF, Slug, Snail, Tamps, target of rapamycin, TGF-β, TIM-1, TLR, TNF-R1, TNF-RII, TNFRSF5, TNF-α, total phospho-Akt, TRACAP, Twist; VCAM-1, VEGF, or vimentin; decreased levels of a protein selected from E-cadherin, cytokeratin, laminin, claudin 1, occludens, PECAM-1, desmin, podocin, and ZO-1; decreased levels of an mRNA that encodes a protein selected from E-cadherin, cytokeratin, laminin, claudin 1, occludin, PECAM-1, desmin, podocin, and ZO-1; and increased nuclear localization of a protein selected from β-catenin and CBF-A.

In some embodiments, physiological changes characteristic of fibrosis include changes in cell or cell culture morphology including but not limited to increased spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, enhanced individual cell migration.

In some embodiments, physiological changes characteristic of fibrosis include changes in cell area, compactness, eccentricity, extent, solidity, angle between neighbors, radial distribution, angular second movement, contrast, difference entropy, difference variance, entropy, inverse difference moment, sum average, sum variance, or variance.

In some embodiments, physiological changes characteristic of fibrosis include an increase in the levels of one or more of the following proteins, for which representative GenBank Accession numbers are shown in parentheses: vimentin (NP 003371.2), fibronectin (AAA53376.1), alpha smooth muscle actin (aSMA; NP 001606.1), connective tissue growth factor (CTGF; AAA91279.1), collagen type IV (col IV; AAD13909.1), collagen type I (col I; AAA60150.1), interleukin 1-beta (IL-1b; AAA74137.1), interleukin 10 (IL-10; CAG46825.1), interleukin 6 (IL-6; CAG29292.1), monocyte chemotactic protein-1 (MCP-1; AAH09716.1), macrophage inflammatory protein-1 alpha (MIP-1a; AAI71831.1), macrophage inflammatory protein-1 beta (MIP-1b; AAX07305.1), RAC-beta serine/threonine-protein kinase (Akt2; AAI20996.1), cyclic AMP response element-binding protein (CREB; AAH10636.1), c-Jun N-terminal kinase 2 (JNK2; CAG38817.1), mitogen-activated protein kinase 6 (MKK6; NP 002749.2), mitogen-activated protein kinase 13 (p38-delta; NP 002745.1), ribosomal S6 kinase 2 (RSK2; NP 004577.1), target of rapamycin (TOR; NP 004949.1), glycogen synthase kinase 3-alpha/beta (GSK-3a/b; NP 063937.2), extracellular signal-regulated kinase (ERK; NP 002736.3), protectin (CD59; CAG46523.1), chitinase-3-like protein 1 (YKL40; AAH39132.1), chemokine (C-X-C motif) ligand 16 (CXCL16; AAQ89268.1), matrix metalloproteinase 9 (MMP9; AAH06093.1), myeloperoxidase (MPO; AAA59896.1), resistin (FIZZ3; AA038860.1), L-selectin (CD62L; AAH20758.1), sialic acid binding immunoglobulin-like lectin 5 (Siglec-5 or CD170; AAH29896.1), tumor necrosis factor receptor 1 (TNFR1; AAA61201.1), tumor necrosis factor receptor 2 (TNFR2; BAA89055.1), alanyl (membrane) aminopeptidase (ANPEP; AAH58928.1), cysteine-rich angiogenic inducer 61 (Cyr61; CAG38757.1), CD10 (NP 009220.2), stem cell factor (SCF or kit-ligand; P21583.1), growth regulated protein alpha (GRO-a; NP 001502.1), dipeptidyl peptidase-4 (DDPIV; NP 001926.2), epidermal growth factor (EGF; NP 001954.2), epidermal growth factor receptor (EGFR; AAH94761.1), fatty acid binding protein 1 (FABP1; CAG46887.1), T-cell immunoglobulin and mucin-domain containing protein 1 (TIM-1; BAJ61033.1), lipoclain-2 (NP 005555.2), tumor necrosis factor alpha (TNFa: CAA78745.1), vascular cell adhesion protein 1 (VCAM1; AAH85003.1), vascular endothelial growth factor (VEGF; CAC19513.2), annexin V (NP 001145.1), angiotensinogen (AAA51679.1), interleukin 1 receptor alpha (IL-1Ra; NP 000868.1), CD40L (NP 000065.1), CD44H (ACI46596.1), lymphocyte function associated antigen 3 (LFA-3; P19256.1), CD99 (CAG29282.1), galectin 1 (NP 002296.1), integrin beta 1 (AAH20057.1), integrin beta 2 (AAH05861.1), and integrin beta 3 (NP 000203.2).

In some embodiments, physiological changes characteristic of fibrosis include a decrease in the levels of one or more of the following proteins, for which representative GenBank Accession numbers are shown in parentheses: zonula occludens-1 (ZO-1; NP 003248.3), heat shock protein 27 (HSP27; BAB17232.1), fetuin A (NP 001613.2), retinol binding protein 4 (RBP4; NP 006735.2), serpin peptidase inhibitor (Serpin A3; AAH03559.3), endothelial cell-selective adhesion molecule precursor (ESAM; NP 620411.2), junctional adhesion molecule C (JAM-C; Q9BX67.1), and vascular adhesion protein-1 (VAP-1; Q16853.3).

As used herein, “trans-well fibrosis assay” refers to the in vitro culture of organ resident cells (e.g., renal cells) in which the cells are not in direct contact with a biological sample from a subject, but are contacted by culture medium that has been in direct contact with the biological sample, and vice versa. Illustrative trans-well methods include culturing renal cells and cells from a subject (e.g., peripheral blood mononuclear cells (PBMCs)) in adjacent culture dishes separated by a semipermeable membrane. According to this method, molecules present in the culture medium of the subject's cells pass through the membrane and contact the organ resident cells. By this method, the organ resident cells are contacted by molecules secreted by the subject's cells without being directly contacted by the subject's cells. Additionally or alternatively, culture media may be manually transferred between the two cultures.

As used herein, short-term diabetes refers to diabetes of less than five year duration. As used herein, long-term diabetes refers to diabetes of greater than 10 years duration.

As used herein, “normoalbuminuria” or “normoA” refers to a urine albumin levels of less than 30 mg/L. As used herein “short-term normoA” refers to type 2 diabetics that have been diagnosed with diabetes for less than 5 years and have urine albumin levels of less than 30 mg/L. As used herein “long-term normoA” refers to type 2 diabetics that have been diagnosed with diabetes for greater than 10 years and have urine albumin levels of less than 30 mg/L.

As used herein, “microalbuminuria” or “microA” refers to urine albumin levels from about 30 mg/L to about 300 mg/L.

As used herein, “macroalbuminuria” refers to urine albumin levels greater than about 300 mg/L. Macroalbuminaria is a characteristic of diabetic nephropathy.

As used herein, “peripheral blood mononuclear cells” or “PBMCs” refers to cells of the peripheral blood having round nuclei, including, for example, lymphocytes, monocytes, macrophages, basophils, and dendritic cells.

As used herein, “insulin resistance” refers to a state or condition in which a subject's body tissues have a lowered level of response to insulin compared to healthy control subjects.

As used herein, “diabetic nephropathy” or “DN” refers to a set of structural and functional changes of the kidney characterized by an accumulation of extracellular matrix proteins that results in a decline in excretory function and scar formation. Clinically, this scarring results in decreased glomerular filtration rate (GFR), increased proteinuria, systemic hypertension, and loss of renal function leading to the need for dialysis and transplantation.

There are no symptoms in the early stages of diabetic nephropathy. The only sign of kidney damage may be small amounts of protein leaking into the urine (microalbuminuria). Normally, protein is not found in urine except during periods of high fever, strenuous exercise, pregnancy, or infection. Diabetic nephropathy can be diagnosed by the presence of microalbuminuria (greater 300 mg albumin/24 hr or an albumin to creatinine ratio (ACR) of 3.4-34 mg/mmol). In addition to the diagnosis of diabetic nephropathy based on the albumin levels, the stage of chronic kidney disease is also noted based on a subject's GFR. Histological changes characteristic of diabetic nephropathy include but are not limited to mesangial expansion, glomerular basement membrane thickening, and glomerular sclerosis. Glomeruli and the kidneys themselves can increase in size, which distinguishes diabetic nephropathy from other forms of chronic kidney disease. In the case of atypical presentation, as is common in patients with type 2 diabetics, a renal biopsy is usually indicated.

As used herein, the terms “treating” or “treatment” or “alleviation” refers to both therapeutic treatment and prophylactic or preventative measures, wherein the object is to prevent or slow down (lessen) the targeted pathologic condition or disorder. It is also to be appreciated that the various modes of treatment or prevention of medical conditions as described are intended to mean “substantial,” which includes total but also less than total treatment or prevention, and wherein some biologically or medically relevant result is achieved.

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein “agents” or “compounds” with the capacity to prevent or treat diabetic nephropathy or other complications of diabetes, diabetes, and/or insulin resistance refers to agents or compounds that reduce, prevent or delay the onset of, reduce the severity of onset of, or ameliorate the symptoms associated with diabetic nephropathy or other complications of diabetes, diabetes, and/or insulin resistance. As used herein, the term encompasses agents that prevent or delay the onset of, reduce the severity of onset of, or suppress the physiological and/or morphological changes characteristic of fibrosis such as those described herein.

As used herein, a “compilation” of biomarkers, physiological changes, or morphological changes refers to a listing of biomarkers, physiological changes, or morphological changes that may be detected by the present methods and are relevant for detecting the presence of or predisposition to diabetes, complications of diabetes (e.g., diabetic nephropathy) and/or insulin resistance according to the present methods. In some embodiments, compilations of biomarkers, physiological changes, or morphological changes are components of kits.

### Methods

In one aspect, the present disclosure provides methods for detecting a presence of or predisposition to diabetes, complications of diabetes (e.g., diabetic nephropathy) or insulin resistance. In some embodiments, the methods comprise co-culturing a biological sample from the subject in vitro with one or more organ resident cells, (e.g. a renal cell line), maintaining the co-culture for a sufficient time for the biological sample to induce physiological changes in the cells, and detecting the physiological changes in the cells.

According to the present methods, organ resident cells (e.g. renal cells) are contacted with a subject's biological sample for a sufficient time for the biological sample to induce physiological changes in the cells that are characteristic of fibrosis. Contact between the cells and the subject's sample may be direct, such as by co-culture, or indirect, such as by culture in adjacent wells separated by a semipermeable membrane (i.e. trans-well culture). In some embodiments, the physiological changes comprise changes in cell or cell culture morphology. In some embodiments, the physiological changes comprise changes in the expression level of one or more biomarkers.

Biological samples may be collected according to methods known in the art or according to illustrative methods described herein. In some embodiments, the biological sample comprises peripheral blood mononuclear cells (PBMCs). In some embodiments, the biological sample comprises urine. In some embodiments, the biological sample comprises saliva, whole blood, serum, plasma, cells, tissues, cerebrospinal fluid, or interstitial fluid. One of skill in the art will understand that collection, handling, and storage of samples will vary according to the particular sample in use and may be optimized with respect to various factors, including operator preferences.

Co-culture of biological samples with organ resident cells (e.g. renal cells) comprises direct contact of the cell line with the subject's cells. Culture conditions may be optimized according to the particular cell types in use and may be optimized according to methods known in the art. Illustrative organ resident cell lines include but are not limited to embryonic and non-embryonic cell lines, renal cell lines derived from human renal proximal tubular epithelial cells, human renal epithelial cell culture model, human renal epithelial cells, or human renal cortical epithelial cells. In some embodiments, the organ resident cell line is a renal cell line. In some embodiments, the renal cell line is HK-2.

Organ resident cells (e.g. renal cells) may also be exposed to a subject's biological sample indirectly, as by trans-well culturing or manual transfer of culture media. According to the trans-well method, organ resident cells are separated from the subject's biological sample by a semipermeable membrane that permits the transfer of molecules secreted by the subject's cells to be transferred to the renal cell line. This method thus permits the operator to distinguish between physiological changes in the organ resident cell line that require direct contact with the subject's biological sample and those that do not.

Organ resident cells (e.g. renal cells) may be cultured using conditions standard in the art, including illustrative conditions described herein. Culture conditions may be optimized according to the particular cells or biological sample in use, as will be understood by one of skill in the art.

In some embodiments, organ resident cells (e.g. renal cells) are contacted with a subject's biological sample for a period sufficient to induce physiological changes characteristic of fibrosis in the cells. In some embodiments, a sufficient time comprises about 12 to about 72 hours. In some embodiments, a sufficient time comprises about 24 to about 48 hours. In some embodiments, a sufficient time comprises about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 hours.

According to the present methods, direct or indirect contact of a subject's biological sample with cultured organ resident cells (e.g. renal cells) induces physiological changes in the cells. In some embodiments, the physiological changes comprise changes in cell or cell culture morphology characteristic of fibrosis. In some embodiments, the changes comprise changes in the level of one or more biomarkers characteristic of fibrosis.

Any suitable aspect of cell or cell culture morphology may be measured in the present methods. Illustrative morphological aspects include but are not limited to spindle formation, cell elongation, cell contractility/mobility, proliferation, apoptosis, necrosis, viability, cell-cell contact, filapodial stress fibers, cytoskeletal organization, number and positioning of tight intercellular junctions, formation of focal adhesions, and individual cell migration.

In some embodiments, morphological changes indicative of fibrosis include but are not limited to increased spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, enhanced individual cell migration.

In some embodiments, physiological changes characteristic of fibrosis include changes in cell area, compactness, eccentricity, extent, solidity, angle between neighbors, radial distribution, angular second movement, contrast, difference entropy, difference variance, entropy, inverse difference moment, sum average, sum variance, or variance.

In some embodiments, morphological changes indicative of fibrosis include but are not limited to an increase in one or more of the following: cell area shape parameters including compactness; cell distribution parameters including radial distribution fractions; and Haralick features of texture parameters including contrast, difference entropy, difference variance, entropy, sum average, sum entropy, sum variance, and variance. In some embodiments, morphological changes indicative of fibrosis include but are not limited to a decrease in one or more of the following: cell area shape parameters including area, extent, and solidity; cell distribution parameters including angle between neighbors, inverse difference and moment.

In some embodiments, physiological changes characteristic of fibrosis include changes in the levels of one or more of vimentin, fibronectin, alpha smooth muscle actin (aSMA), connective tissue growth factor (CTGF), collagen type IV (col IV), collagen type I (col I), interleukin 1-beta (IL-1b), interleukin 10 (IL-10), interleukin 6 (IL-6), monocyte chemotactic protein-1 (MCP-1), macrophage inflammatory protein-1 alpha (MIP-1a), macrophage inflammatory protein-1 beta (MIP-1b), RAC-beta serine/threonine-protein kinase (Akt2), cyclic AMP response element-binding protein (CREB), c-Jun N-terminal kinase 2 (JNK2), mitogen-activated protein kinase 6 (MKK6), mitogen-activated protein kinase 13 (p38 delta), ribosomal S6 kinase 2 (RSK2), target of Rapamycin (TOR), glycogen synthase kinase 3 alpha/beta (GSK-3a/b), extracellular signal-regulated kinase (ERK), protectin (CD59), chitinase-3-like protein 1 (YKL40), chemokine (C-X-C motif) ligand 16 (CXCL16), matrix metalloproteinase 9 (MMP9), myeloperoxidase (MPO), resistin (FIZZ3), L-selectin (CD62L), sialic acid binding immunoglobulin-like lectin 5 (Siglec-5 or CD170), tumor necrosis factor receptor 1 (TNFR1), tumor necrosis factor receptor 2 (TNFR2), alanyl (membrane) aminopeptidase (ANPEP), cysteine-rich angiogenic inducer 61 (Cyr61), CD10, stem cell factor (SCF or kit-ligand), growth regulated protein alpha (GRO-a), dipeptidyl peptidase-4 (DDPIV), epidermal growth factor (EGF), epidermal growth factor receptor (EGFR), fatty acid binding protein 1 (FABP1), T-cell immunoglobulin and mucin-domain containing protein 1 (TIM-1), lipoclain-2, tumor necrosis factor alpha (TNFa), vascular cell adhesion protein 1 (VCAM1), vascular endothelial growth factor (VEGF), annexin V, angiotensinogen, interleukin 1 receptor alpha (IL-1Ra), CD40L, CD44H, lymphocyte function associated antigen 3 (LFA-3), CD99, galectin 1, integrin beta 1, integrin beta 2, and integrin beta 3, zonula occludens-1 (ZO-1), heat shock protein 27 (HSP27), fetuin A, retinol binding protein 4 (RBP4), serpin peptidase inhibitor (Serpin A3), ESAM, JAM-C, or VAP-1.

Physiological changes associated with fibrosis may be detected using methods known in the art as appropriate to the physiological change in question. Changes in cell or cell culture morphology may be assessed visually using any suitable form of microscopy, such as but not limited to light microscopy, fluorescence microscopy, or electron microscopy. Samples will be prepared in accordance with the type of microscopy in use. For example, cells may be viewed as unstained, non-specifically stained, or specifically stained samples. In some embodiments, morphological changes are detected using light microscopy with samples left unstained. In some embodiments, morphological changes are detected using fluorescence microscopy with cells stained specifically for F-actin, ZO-1, and DNA.

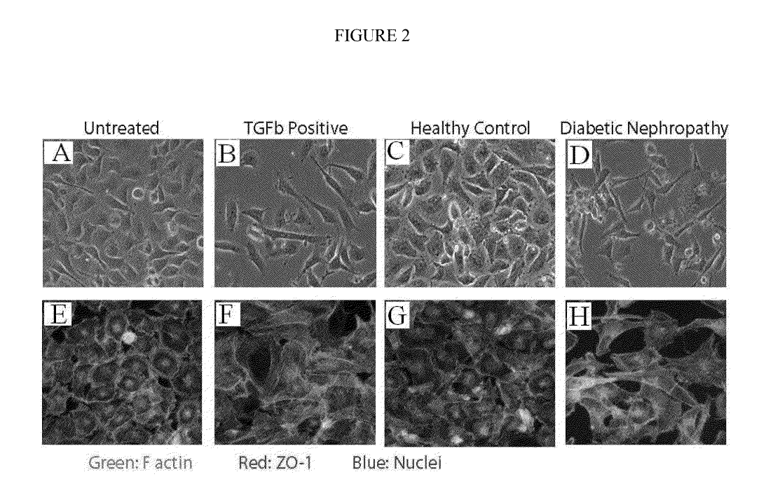

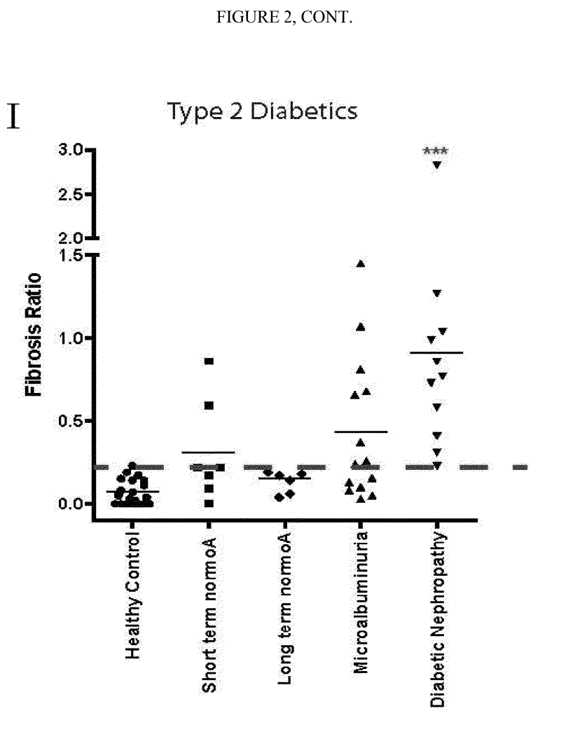

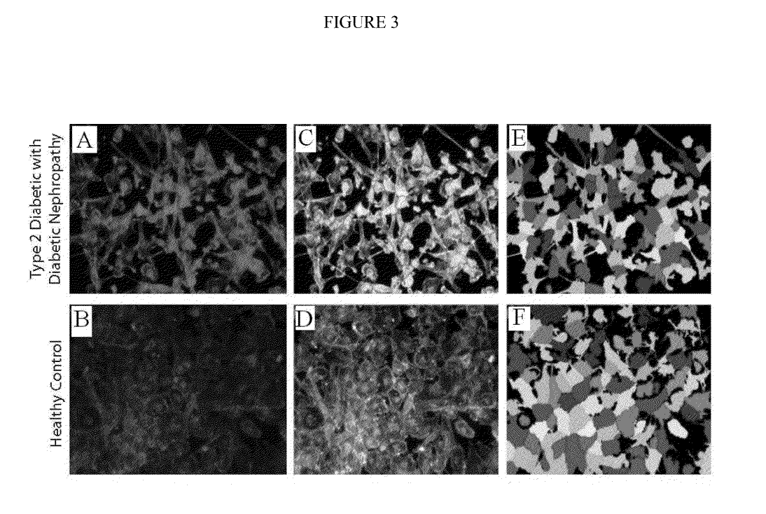

Changes in cell or cell culture morphology may be detected manually, such as by the manual observation and quantification by the operator. Illustrative results from manual detection of changes in cell or cell culture morphology are presented in the examples below and in FIG. 2 and FIG. 3.

Additionally or alternatively, changes in cell or cell culture morphology may be assessed using computer-assisted methods such as described in the examples below. Illustrative results from computer-assisted detection of changes in cell or cell culture morphology are presented in the examples below and in FIG. 3 and Table 1.

Detection of changes in the levels of biomarkers may be performed using methods known in the art, including but not limited to quantification of immunostaining, protein array analysis, ELISA, western blotting, flow cytometry for the detection of changes in protein levels. One of skill in the art will understand that what constitutes the most suitable method will depend on the particular biomarker being detected, available reagents, and available equipment. Illustrative results of detecting biomarkers using immunostaining, protein array analysis, western blotting, and ELISA are presented in the examples below.

Additionally or alternatively changes the levels of biomarkers may be measured on the RNA level using methods known in the art including but not limited to RT-PCR, northern blotting, and in situ hybridization. Whether changes in biomarker levels are assessed on the level of protein or RNA will depend on the particular biomarker being detected, available reagents, and available equipment.

One of skill in the art will understand that results of the present methods must be interpreted in view of results obtained for one or more suitable controls. One of skill will further understand that what constitutes a suitable control will depend on a number of factors, including the characteristics of the subject in question and the precise determination being made. For example, where the subject is not a known diabetic and the determination to be made is the presence of or predisposition to insulin resistance, a suitable controls include biological samples from a subject known to be insulin resistant as well as biological samples from a healthy subject. Similarly, where the subject is a known diabetic and the determination is to be made of the presence of or predisposition to diabetic nephropathy, suitable controls would include biological samples from a diabetic subject with nephropathy and from a diabetic subject without nephropathy. Illustrative controls for various determinations are provided in the examples below. In some embodiments, the control is a subject having no known insulin resistance, diabetes, or complications of diabetes (i.e. a healthy control). In some embodiments, the control is a DM2 subject. In some embodiments, the control is a DM2 subject having normal kidney function (i.e. a healthy control). In some embodiments, the control is a type 2 diabetic with nephropathy.

In another aspect, the present disclosure provides a method for identifying agents or compounds with the capacity to regulate (e.g., directly or indirectly) physiological and/or morphological changes characteristic of fibrosis. By “capacity to regulate” is meant the capacity to induce, augment, suppress, reduce, or otherwise influence the extent or severity of physiological and/or morphological changes characteristic of fibrosis. In some embodiments, the methods comprise co-culturing a biological sample from a subject in vitro with one or more organ resident cell lines (e.g. renal cell lines), maintaining the co-culture for a sufficient time for the biological sample to induce physiological changes in the cells, contacting the cells with a candidate agent, detecting physiological changes in the cells, and comparing the physiological changes to those of a control sample. In some embodiments, the methods comprise contacting the subject's biological sample with the agent prior to co-culturing the biological sample with one or more organ resident cell lines (e.g. renal cell lines), maintaining the co-culture for a sufficient time for the biological sample to induce physiological changes in the cells, and comparing the physiological changes to those of a control sample. In some embodiments, the biological sample comprises the subject's PBMCs.

Methods of screening described herein comprise embodiments described above with respect to the use of organ resident cells, biological samples, direct versus indirect contact, sufficient time, physiological chances characteristic of fibrosis, detection of physiological changes characteristic of fibrosis, and controls.

Any candidate agent may be screened, including but not limited to antibodies, proteins, metals, salts, small molecules, nanoparticles, or combinations or derivatives thereof. The capacity of a candidate agent to prevent as opposed to treat or reverse physiological changes characteristic of fibrosis will depend on the precise methods used for screening. The capacity of an agent to prevent physiological and/or morphological changes characteristic of fibrosis may be assessed by contacting the organ resident cells (e.g. renal cells) with the agent prior to contact with a biological sample shown to induce the physiological changes. The capacity of an agent to treat or reverse physiological changes characteristic of fibrosis may be assessed by contacting the organ resident cells with a biological sample shown to induce the physiological changes, determining that the changes have occurred, contacting the cells with the agent, and assessing the extent to which the physiological changes are arrested or reversed.

In some embodiments, the biological sample comprises peripheral blood mononuclear cells (PBMCs). In some embodiments, the biological sample comprises urine. In some embodiments, the biological sample is from a subject diagnosed as having type 1 diabetes, type 2 diabetes, insulin resistance, normalbuminuria, microalbuminuria, or macroalbuminuria.

In some embodiments, a sufficient time comprises about 12 to about 72 hours. In some embodiments, a sufficient time comprises about 24 to about 48 hours. In some embodiments, a sufficient time comprises about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 hours.

In some embodiments, the physiological changes comprise changes in cell or cell culture morphology. In some embodiments, the changes in cell or cell culture morphology comprise changes associated with fibrosis.

In some embodiments, the changes in cell or cell culture morphology associated with fibrosis comprise one or more of spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, enhanced individual cell migration.

In some embodiments, the changes in cell or cell culture morphology comprise changes in cell area, compactness, eccentricity, extent, solidity, angle between neighbors, radial distribution, angular second movement, contrast, difference entropy, difference variance, entropy, inverse difference moment, sum average, sum variance, or variance.

In some embodiments, the physiological changes comprise changes in protein levels. In some embodiments, the changes in protein level comprise an increase in one or more of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII. IL-1β, IL10, MIP-1α, MIP-1β, phospho-CREB, DPPIV, EGF, EGFR, TIM-1, TNF-α, VEGF, annexin V, angiotensin, CXCL16, MCP-1, GRO-α, or IL-1Ra. In some embodiments, the changes in protein level comprise a decrease in one or more of phospho-HSP27, JAM-C, podocalyxin, and VAP-1.

In some embodiments, the detecting comprises microscopy, immunostaining, ELISA, protein arrays, western blotting or flow cytometry. In some embodiments, the renal cells are contacted with the agent prior to, simultaneous to, or subsequent to culturing in conjunction with the biological sample.

### Kits

In one aspect, the present disclosure provides a kit for determining a presence of or predisposition to diabetic nephropathy, diabetes, and/or insulin resistance in a subject, comprising a means to co-culture a biological sample with an organ resident cell line (e.g. a renal cell line), a compilation of biomarkers, a compilation of physiological changes predictive of the presence of or predisposition to diabetic nephropathy, diabetes, and/or insulin resistance, and instructions for use.

In some embodiments, the compilation of physiological changes predictive of the presence of or predisposition to diabetic nephropathy, diabetes, and/or insulin resistance comprises a compilation of parameters describing cell or cell culture morphology.

In some embodiments, the parameters describing cell or cell culture morphology comprise parameters relating to one or more of spindle formation, cell elongation, cell contractility/mobility, proliferation, apoptosis, necrosis, viability, cell-cell contact, filapodial stress fibers, cytoskeletal reorganization, tight intercellular junctions, focal adhesions, individual cell migration.

In some embodiments, the parameters describing cell or cell culture morphology comprise parameters relating to one or more of cell area, compactness, eccentricity, extent, solidity, angle between neighbors, radial distribution, angular second movement, contrast, difference entropy, difference variance, entropy, inverse difference moment, sum average, sum variance, or variance.

In some embodiments, the compilation of physiological changes predictive of the presence of or predisposition to diabetic nephropathy, diabetes, and/or insulin resistance comprises a compilation of predictive biomarkers.

In some embodiments, the compilation of predictive biomarkers comprises one or more of vimentin, fibronectin, alpha smooth muscle actin (aSMA), connective tissue growth factor (CTGF), collagen type IV (col IV), collagen type I (col I), interleukin 1-beta (IL-1b), interleukin 10 (IL-10), interleukin 6 (IL-6), monocyte chemotactic protein-1 (MCP-1), macrophage inflammatory protein-1 alpha (MIP-1a), macrophage inflammatory protein-1 beta (MIP-1b), RAC-beta serine/threonine-protein kinase (Akt2), cyclic AMP response element-binding protein (CREB), c-Jun N-terminal kinase 2 (JNK2), mitogen-activated protein kinase 6 (MKK6), mitogen-activated protein kinase 13 (p38 delta), ribosomal S6 kinase 2 (RSK2), target of Rapamycin (TOR), glycogen synthase kinase 3 alpha/beta (GSK-3a/b), extracellular signal-regulated kinase (ERK), protectin (CD59), chitinase-3-like protein 1 (YKL40), chemokine (C-X-C motif) ligand 16 (CXCL16), matrix metalloproteinase 9 (MMP9), myeloperoxidase (MPO), resistin (FIZZ3), L-selectin (CD62L), sialic acid binding immunoglobulin-like lectin 5 (Siglec-5 or CD170), tumor necrosis factor receptor 1 (TNFR1), tumor necrosis factor receptor 2 (TNFR2), alanyl (membrane) aminopeptidase (ANPEP), cysteine-rich angiogenic inducer 61 (Cyr61), CD10, stem cell factor (SCF or kit-ligand), growth regulated protein alpha (GRO-a), dipeptidyl peptidase-4 (DDPIV), epidermal growth factor (EGF), epidermal growth factor receptor (EGFR), fatty acid binding protein 1 (FABP1), T-cell immunoglobulin and mucin-domain containing protein 1 (TIM-1), lipoclain-2, tumor necrosis factor alpha (TNFa), vascular cell adhesion protein 1 (VCAM1), vascular endothelial growth factor (VEGF), annexin V, angiotensinogen, interleukin 1 receptor alpha (IL-1Ra), CD40L, CD44H, lymphocyte function associated antigen 3 (LFA-3), CD99, galectin 1, integrin beta 1, integrin beta 2, and integrin beta 3, zonula occludens-1 (ZO-1), heat shock protein 27 (HSP27), fetuin A, retinol binding protein 4 (RBP4), serpin peptidase inhibitor (Serpin A3), ESAM, JAM-C, and VAP-1.

In some embodiments, the kit comprises one or more positive or negative control biological samples.

### EXAMPLES

The following examples are presented in order to more fully illustrate the embodiments of the present technology. These examples should in no way be construed as limiting the scope of the invention, as defined by the appended claims.

### Materials and Methods

### Preparation of Human Peripheral Blood Lymphocytes

Blood samples were collected in a yellow-top acid citrate dextrose (ACD) tube, and centrifuged for 10 minutes. Plasma was transferred to a sterile conical tube and stored at −20° C. until use. The remaining blood fraction was diluted 1:2 with sterile Dulbecco's phosphate buffered saline (DPBS). 25 mL of the diluted blood fraction was layered over 15 mL of lymphocyte separation medium (LSM; Cellgro®, Mediatech, Inc., Manassas, Va., USA) in a 50 mL tube without mixing. The sample was then centrifuged for 30 minutes at 1340 rpm (410×g, without brake). The white blood cells (WBCs) were collected, washed 3× in sterile DPBS, centrifuged for 10 minutes at 1200 rpm (330×g, with brake) following each wash, and re-suspended in assay medium or stored frozen until use. For frozen storage, cells were re-suspended in freezing media (10% DMSO in fetal calf serum) at 10 million cells/ml freezing media, placed at −80° C. for 24 hours, and transferred to liquid nitrogen. For use, cells were thawed rapidly under warm water and placed immediately into 9 ml of thaw solution (50% RPMI+50% fetal calf serum). Cells were centrifuges for 5-10 minutes at 1200 rpm, without brake, washed 3× with sterile DPBS, and re-suspended in cell culture medium.

### Preparation of Human Serum

Blood samples were collected in red-top or gold-top serology tube, and centrifuged for 10 minutes. Serum was transferred to a sterile conical tube and stored at −20° C. until use.

### Cell Culture

Cell line HK-2 (human renal proximal tubular epithelial cells; ATCC CRL-2190) was maintained in keratinocyte serum-free media (Keratinocyte-SFM; Invitrogen, Grand Island, N.Y., USA) supplemented with penicillin/streptomycin (100 U/ml), and 2 mM glutamine, with or without 1% FCS.

Cell line A549 (human alveolar epithelial cells; ATCC CCL-185) was maintained in F-12K serum-free media (ATCC) supplemented with 10% FCS, penicillin/streptomycin (100 U/ml), and glutamine (2 mM).

Fibrosis Assay with HK-2 Cells

HK-2 cells were plated in keratinocyte SFM on gelatin-coated coverslips (0.1% gelatin) in 24 well plates. Cells were plated at 1×10⁵ cells per well in 1 ml of medium and incubated overnight. Human PBMCs were re-suspended at 1×10⁶/ml in HK-2 medium and added to the wells in a total volume of 1 ml. TGF-β1 (20 ng/ml; Sigma-Aldrich, St. Louis, Mo., USA) was added to some wells as a positive control for fibrosis. See Zeisberg et al. J Clin Invest. 2009; 119(6):1429-1437. For assay of purified lymphocytes, the cells were re-suspended at 8×10⁴/ml. Under some conditions, HK-2 cells were exposed to a subject's urine. Under some conditions, cells were maintained in a 0.4 μm trans-well culture system (BD Biosciences, San Jose, Calif., USA). Under some conditions, PBMCs/HK-2 co-cultures included anti-cytokine antibodies or MAPK pathway inhibitors.

After 24 hours of PBMC co-culture, supernatants were collected and stored at −20° C. until analysis. After thawing, cells were washed 2× with PBS, fixed with 4% paraformaldehyde for 10 minutes, washed 3× with PBS and analyzed using phase-contrast microscopy and immunofluorescence.

Fibrosis Assay with A549 Cells

A549 cells were plated in complete F-12K medium on gelatin-coated coverslips in 24 well plates. Cells were plated at 5×10⁴ cells per well in 1 ml of medium. 24 hours later, human PBMCs were re-suspended at 1×10⁶/ml in complete F-12K medium and added to the wells in a total volume of 1 ml. TGF-β1 (20 ng/ml; Sigma) was added to some wells as a positive control for fibrosis. After 24 hours, supernatants were collected and analyzed. Cells were washed 2× with PBS, fixed with 4% paraformaldehyde for 10 minutes, washed 3× with PBS and analyzed using phase microscopy and immunofluorescence.

### Fibrosis Assay in 100 mm Plates for Protein Expression Analysis

HK-2 cells were plated in keratinocyte SFM in 100 mm plates. Cells were plated at 1×10⁶ cells per plate in 10 ml of medium. 48 hours later, human PBMCs were re-suspended in HK-2 medium and added to the plates at 3×10⁶ PBMCs in a total volume of 10 ml. TGF-01 (20 ng/ml; Sigma) was added to some plates as a positive control for fibrosis. See Zeisberg et al. J Clin Invest. 2009; 119(6):1429-1437. After 48 hours, supernatants were collected and analyzed using flow cytometry. Cells were washed 2× in PBS and 0.5 ml of cell lysis buffer (RIPA+HALT protease inhibitor, Pierce, Rockford, Ill., USA) was added to the plate to lyse the cells. A cell scraper was used to aid in cell lysis. Cell lysates were collected and analyzed using by western blot and proteome arrays.

### Fibrosis Assay for Phospho-ELISA Analysis

HK-2 cells were plated in keratinocyte SFM in 6 well plates. Cells were plated at 3×10⁵ cells per well in 3 ml of medium. 24 hours later, human PBMCs were re-suspended in HK-2 medium and added to the plates at 2×10⁶ PBMCs in a total volume of 3 ml. TGF-β1 (20 ng/ml; Sigma) was added to some plates as a positive control for fibrosis. See Zeisberg et al. J Clin Invest. 2009; 119(6):1429-1437. After 24 hours, HK-2 cells were washed 2× in PBS. HK-2 cell lysate was made and phosphorylated MAPK proteins were analyzed using phospho-ELISA kits for ERK, p38, and JNK according to manufacturer's directions (eBioscience).

### Western Blot

A bicinchoninic acid (BCA) protein assay (Pierce) was performed on cell lysates to determine total protein concentration and 10-50 μg of protein was run per sample. Cell lysate samples were prepared to run under reducing and denaturing conditions with NuPage® LDS sample buffer and NuPage® Reducing Agent (Invitrogen). MOPS SDS Running Buffer with antioxidant was used to run the gel (Invitrogen). Pre-cast 4-12% Bis-Tris mini gels were used for western blotting (Invitrogen). Gels were transferred to PVDF membrane using the iBlot® Transfer System according to manufacturer's directions (Invitrogen, Grand Island, N.Y., USA). Membranes were treated with SuperSignal Western Blot Enhancer according to manufacturer's directions (Thermo Scientific, Waltham, Mass., USA). Membranes were blocked with 5% milk in PBS for 30 minutes, followed by overnight incubation with primary antibody at 4° C. on a rocker. Membranes were washed 4× in PBS+0.5% Tween, followed by incubation with secondary antibody for 1 hour at room temperature or overnight at 4° C. on a rocker. Membranes were washed 4× in PBS+0.5% Tween and developed using SuperSignal West Femto Maximum Sensitivity Substrate (Thermo Scientific) and analyzed using a LAS-4000 mini analyzer (FujiFilm). Protein was quantified by band intensity relative to the housekeeping protein GAPDH using the software ImageJ.

Primary antibodies used were aSMA (clone 1A4, Sigma), collagen type IV clone COL-94 (Sigma), collagen type I clone COL-1 (Sigma), CTGF (Pierce), E-cadherin clone 36/E-cadherin (BD), fibronectin clone FN-15 (Sigma), vimentin (Sigma) and GAPDH clone GAPDH-71.1 (Sigma). Secondary antibodies used were goat anti-rat HRP (Invitrogen) and rabbit anti-mouse IgG+IgM peroxidase conjugated (Thermo Scientific).

### Flow Cytometric Analysis of Cytokine and Chemokine Production

Cytokine and chemokine levels were measured in the culture supernatant using Flow Cytomix Human Chemokine 6plex, Human Th1/Th2/Th9/Th17/Th22 13plex Kit FlowCytomix, and Human TGF-β1 FlowCytomix Simplex kit according to manufacturer's directions (eBioscience, San Diego, Calif., USA). Analysis was performed using a FacsCalibur™ flow cytometer (BD Biosciences) and the FlowCytoMix PRO software (eBioscience).

### Immunofluorescence

Cells were permeabilized with 0.5% Triton X-100 in PBS followed by blocking with 1% BSA for 30 minutes at room temperature. Cells were incubated with primary antibodies for 1 hour at 37° C. Cells were washed 3× in PBS and incubated with secondary antibody (when needed) for 1 hour at 37° C. Cells were washed 3× in PBS and the coverslips were mounted to slides with ProLong Gold plus DAPI (Invitrogen) and allowed to cure overnight before microscopic analysis. Primary antibodies used were aSMA (clone 1A4, Sigma), collagen type IV clone COL-94 (Sigma, St. Louis, Mo.), collagen type I clone COL-1 (Sigma), CTGF (Pierce, Rockford, Ill.), Alexa Fluor 546 phalloidin (Invitrogen, Carlsbad, Calif.), Alexa Fluor 488 phalloidin (Invitrogen, Carlsbad, Calif.), vimentin Cy3 conjugate clone V9 (Sigma), ZO-1 (BD), and ZO-1 594 (Invitrogen). Secondary antibodies used were goat anti-mouse IgG, IgM Alexa Fluor 488 (Invitrogen) and goat anti-rat Alexa Fluor 546 (BD).

### Proteome Arrays

Samples were analyzed for select proteins using proteome profiler arrays for kidney biomarkers, apoptosis, non-hematopoietic soluble receptors, hematopoietic soluble receptors, and phosphorylated MAP kinases according to manufacturer's instructions (R&D Systems, Minneapolis, Minn., USA). Samples tested include: urine, plasma, sera, PBMCs lysate pre- and post-co-culture in fibrosis assay, HK-2 cell lysate pre- and post-co-culture in fibrosis assay, and supernatant resulting from co-culture of fibrosis assay.

### Fibrosis Ratio Calculation

Fibrosis ratios are given as the ratio of fibrotic cells to normal cells per microscopic field of view minus the background fibrosis ratio, which is the ratio of fibrotic cells to normal cells in the untreated control for that experiment. In one embodiment, the number of cells per 200× microscopic field after 24 hour co-culture was determined.

### Computational Analysis of Morphological Changes in the Fibrosis Assay

The fibrosis ratios calculated using manual counting and images generated from the fibrosis assay were further supported using a computational morphological analysis software, in this case, CellProfiler. Using the images generated by fluorescent microscopy (e.g., with F-actin (red) and nuclei (blue) staining), the computer program determines the shape of a cell, as illustrated in FIG. 3.

Culture of PBMC with Renal Cell Sonicate for Biomarker Analysis:

Cultured HK-2 cells are trypsinized with 0.05% Trypsin with EDTA (Invitrogen), centrifuged, and re-suspended in complete KSF media+1% FBS. A protease inhibitor is added to the HK-2 cells prior to sonication (HALT, Pierce, Rockford, Ill., USA). Cells are sonicated using a VR50 sonicator fitted with a 2 mm probe. The disrupted HK-2 cells are centrifuged for 20 minutes at 14,000×g to remove debris. The total protein concentration of the sonicates is determined using a microBCA Protein Assay kit (Pierce). PBMC are cultured with HK-2 cell sonicate or in complete KSF-media+1% FBS. Supernatant is collected and the PBMC are then analyzed for surface biomarker expression using the Human Cell Surface Marker Screening Panel BD Lyoplate (BD). PBMC are also made into a lysate using RIPA+HALT (Pierce). Lysates and supernatants are analyzed for biomarker expression using proteome arrays and flow cytometry.

### Example 1

### Co-Culture Fibrosis Assay of the Present Technology

A schematic representation of the co-culture fibrosis assay for detection of diabetes, complications of diabetes (e.g., diabetic nephropathy, diabetic retinopathy, cardiovascular disease) and/or insulin resistance is shown in FIG. 1. According to the methods, a biological sample from a subject to be tested is co-cultured with an established renal or epithelial cell line. In some embodiments, the biological sample comprises PBMCs, urine, serum, plasma, or PBMC components. In some embodiments, the established renal or epithelial cell line is in direct contact with the subject's biological sample, such as where the subject's PBMCs are co-cultured with the cell line. The renal or epithelial cell line is cultured in the presence of the subject's biological sample for a sufficient time for the sample to induce physiological changes in the cell line and/or the biological sample, including but not limited to morphological changes associated with fibrosis and changes in the levels of select proteins or protein phosphorylation. In some embodiments, the morphological changes include but are not limited to spindle formation, extracellular matrix production, and changes in fibrotic markers. In some embodiments, changes in protein level or phosphorylation include changes in the level of resistin and the levels of MAP kinase-mediated phosphorylation. Data resulting from analysis of a number of fibrosis assays are shown in FIG. 2-17 and Tables 1 and 2.

In addition to analysis of the renal cells, the PBMC are analyzed for changes in biomarker levels after culture with the renal epithelial cells or renal cell sonicate. PBMCs are analyzed as whole cells, sonicate, or fractionated sonicate.

### Example 2

### PBMC Co-Culture Fibrosis Assay of the Present Technology Distinguishes Type 1 And 2 Diabetics with and without Diabetic Nephropathy from Healthy Controls

PBMCs from healthy subjects or subjects with type 2 diabetes and nephropathy, microalbuminuria, or normoalbuminuria were co-cultured with HK-2 cells in the co-culture fibrosis assay described in Example 1. Results are shown in FIG. 2. Nephropathy is defined as comprising macroalbuminuria, stage 1V diabetic kidney disease, or diabetic end-stage renal disease.

Morphological and Physiological Changes—

Untreated HK-2 cells showed little to no change in morphology, remaining round and intact, and in the classic cobblestone morphology, suggesting minimal fibrosis induction (FIG. 2A). Addition of TGF-β, which is known to induce epithelial phenotypic and fibrotic changes in HK-2 cells, resulted in fibrotic changes indicated by spindle-like morphology and migration of the cells away from each other (FIG. 2B). Addition of PBMCs from healthy controls to the HK-2 cells caused few or no fibrotic changes (FIG. 2C). By contrast, PBMCs from type 2 diabetics with nephropathy induced fibrotic changes such as spindle formation and “spreading” of the cells (FIG. 2D).

Morphological changes and changes in protein expression were evident when the cells were stained with antibodies directed against F-actin (green) and ZO-1 (red). Untreated HK-2 cells displayed a normal distribution of F-actin around the cell periphery and in cell junctions, and with ZO-1 localized to cell-cell junctions (FIG. 2E). Cells treated with TGF-β displayed morphological changes associated with fibrosis, including increased F-actin spindle formation and re-organization of F-actin into filapodial stress fibers FIG. 2F). These cells also lost the cobblestone morphology, migrated away from each other, and down-regulated ZO-1 expression. Similar fibrotic morphologies were evident in HK-2 cells co-cultured with PBMCs from a type 2 diabetic with nephropathy (FIG. 2H). By contrast, HK-2 cells treated with PBMCs from a healthy control subject did not display fibrotic morphologies (FIG. 2G).

Fibrosis Ratios—

Co-culture fibrosis assays were performed as described in Example 1 using HK-2 cells co-cultured with PBMC cells from the following subjects: (1) healthy controls; (2) short-term (less than 5 years) type 2 diabetic subjects with normoalbuminuria; (3) long-term (greater than 10 years) type 2 diabetic subjects with microalbuminuria; and (4) type 2 diabetics with nephropathy. PBMCs were co-cultured with HK-2 cells for 24 hours. Fibrosis ratios were calculated as the ratio of HK-2 cells displaying phenotypic changes to those displaying no phenotypic changes per 200× microscopic field. The fibrosis ratio of the untreated control is subtracted from ratios calculated for diabetic subjects. FIG. 2C shows the fibrosis ratios for healthy controls (circles; n=20), short-term type 2 diabetic subjects with normoalbuminuria (short-term normoA; squares; n=7)), long-term type 2 diabetic subjects with normoalbuminuria (long-term normoA; diamonds; n=6), type 2 diabetics with microalbuminuria (triangles; n=14), and type 2 diabetics with nephropathy (inverted triangles; n=11). Each dot represents the mean fibrosis ratio calculated for an individual. The p value is <0.0001 for type 2 diabetics with nephropathy versus healthy control subjects and versus long-term type 2 diabetics with normoalbuminuria. The fibrosis ratio data is representative of at least 3 independent experiments. Fibrosis ratio values falling above the dashed line are considered positive ratios, while values falling below the dashed line are considered negative ratios.

These results show that fibrosis ratios are useful for distinguishing between healthy subjects and subjects with diabetic nephropathy. The results further show that fibrosis ratios are useful to distinguish between type 2 diabetics with microalbuminuria, and early indicator of diabetic nephropathy, from healthy subjects.

The results further show that fibrosis ratios are useful to distinguish between short-term type 2 diabetics with normoalbuminuria, who may or may not develop diabetic neuropathy, and long-term type 2 diabetics with normoalbuminuria, who generally do not develop diabetic neuropathy.

As shown in FIG. 2C, long-term type 2 diabetics with normoalbuminuria have similar fibrosis rations to healthy controls, while two of seven short-term type 2 diabetics with normoalbuminuria had higher fibrosis ratios than healthy controls. This fraction correlates with what is known in the art about the frequency with which short-term type 2 diabetics with normoalbuminuria develop nephropathy. In particular, that 30-40% of diabetics progress to nephropathy. These results show that the fibrosis assay of the present methods is predictive of the development of diabetic nephropathy.

Computer-Assisted Analysis—

Fibrosis ratios described were also determined using the CellProfiler morphological analysis software as described in the Materials and Methods. Using the images generated by fluorescent microscopy with F-actin (red) and nuclei (blue) staining, the computer program determined the shape of the cells, as illustrated in FIG. 3. A fibrosis assay was performed as described in Example 1 using HK-2 cells and PBMC from a type 2 diabetic with nephropathy. Cells were stained as after 24 hours of co-culture. FIG. 3A shows HK-2 cells co-cultured with PBMC from a type 2 diabetic with nephropathy, and FIG. 3B shows HK-2 cells after incubation with PBMC from a healthy control. After staining, images were analyzed using the CellProfiler, which quantifies phenotypical changes between the samples. Of the 60 morphological features that were analyzed using CellProfiler, Table 1 displays morphological characteristics that were found to be significantly different between images resulting from the fibrosis assay with PBMC from healthy controls (n=10) and type 2 diabetics with nephropathy (n=10).

TABLE 1
Cell Profiler Morphological Analysis of Images from Fibrosis Assay

| | Healthy Control | Diabetic Nephropathy | |
|---|---|---|---|
| | (n = 10) | (n = 10) | p value |
| Mean Cells Area Shape | | | |
| Area | 8168 ± 1292 | 6707 ± 1935 | 0.046 |
| Compactness | 1.35 ± 0.05 | 144 ± 0.10 | 0.013 |
| Eccentricity | 0.73 ± 0.02 | 0.75 ± 0.02 | 0.053 |
| Extent | 0.54 ± 0.02 | 0.51 ± 0.03 | 0.003 |
| Solidity | 0.78 ± 0.02 | 0.74 ± 0.04 | 0.009 |
| Mean Cells Distribution | | | |
| Angle between neighbors | 114 ± 3.4 | 107 ± 4.9 | 0.0005 |
| Radial Distrib. Mean Fraction 2 of 4 | 1.07 ± 0.06 | 1.15 ± 0.10 | 0.025 |
| 3 of 4 | 1.00 ± 0.02 | 1.06 ± 0.06 | 0.005 |
| 4 of 4 | 0.98 ± 0.02 | 0.94 ± 0.04 | 0.007 |
| Radial Distribution Radial CV 4 of 4 | 0.21 ± 0.02 | 0.26 ± 0.05 | 0.024 |
| Haralick Features - Mean Cells Texture | | | |
| Angular Second Moment | 0.11 ± 0.02 | 0.08 ± 0.02 | 0.001 |
| Contrast | 0.98 ± 0.23 | 1.43 ± 0.37 | 0.002 |
| Difference Entropy | 0.99 ± 0.08 | 1.12 ± 0.10 | 0.001 |
| Difference Variance | 0.57 ± 0.10 | 0.79 ± 0.18 | 0.001 |
| Entropy | 2.78 ± 0.18 | 3.09 ± 0.20 | 0.0007 |
| Inverse Difference Moment | 0.72 ± 0.03 | 0.67 ± 0.04 | 0.004 |
| Sum Average | 6.11 ± 0.68 | 7.49 ± 1.30 | 0.005 |
| Sum Entropy | 2.18 ± 0.10 | 2.35 ± 0.12 | 0.0007 |
| Sum Variance | 7.40 ± 1.54 | 10.32 ± 2.33 | 0.002 |
| Variance | 2.09 ± 0.42 | 2.93 ± 0.64 | 0.001 |

Values shown mean ± standard deviation. P value analyzed using a two tailed students t test with equal variance.

Organ-Resident Cell Type Specificity—

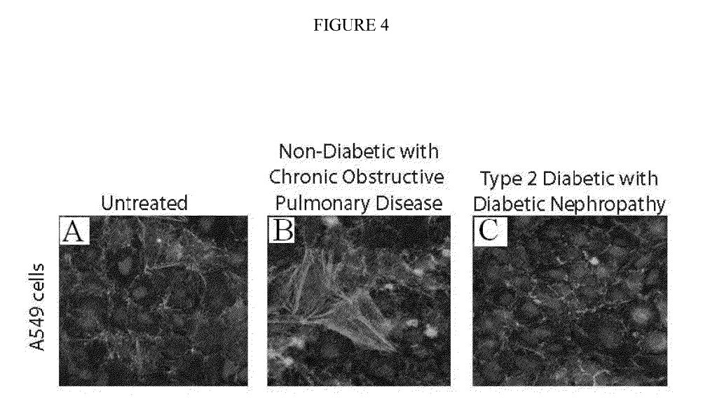

To determine whether the capacity of PBMCs from a type 2 diabetic with nephropathy to induce fibrotic morphologies in renal cells is specific for that cell type, a fibrosis assay was also performed using the A549 human lung epithelial cell line. This cell line is known to behave similarly to HK-2 cells in culture, with the ability to undergo phenotypic and fibrotic changes when appropriately challenged. Results are shown in FIG. 4.

Untreated A549 cells showed normal levels and distribution of ZO-1 and F-actin (FIG. 4A), as did cells co-cultured with PBMCs from a type 2 diabetic with nephropathy (FIG. 4BC). By contrast, A549 cells co-cultured with PBMCs from a subject with chronic obstructive pulmonary disease (COPD) showed morphological changes similar to those shown in FIG. 2A, including spindle formation, F-actin redistribution, ZO-1 down-regulation (FIG. 4B). Notably, COPD is a disorder that affects lung epithelial cells but not renal cells.

These results demonstrate that PBMCs have a specific capacity to induce fibrotic morphologies in cell types derived from organs that are compromised in the individual from which they were isolated, as opposed to a general capacity to induce fibrotic morphologies in all cultured cells.

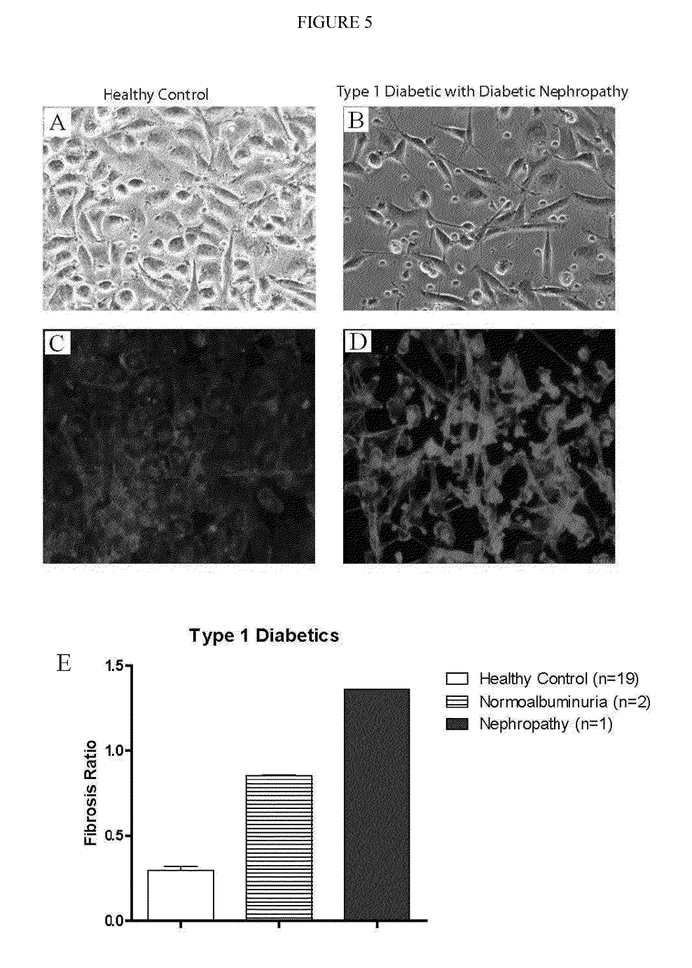

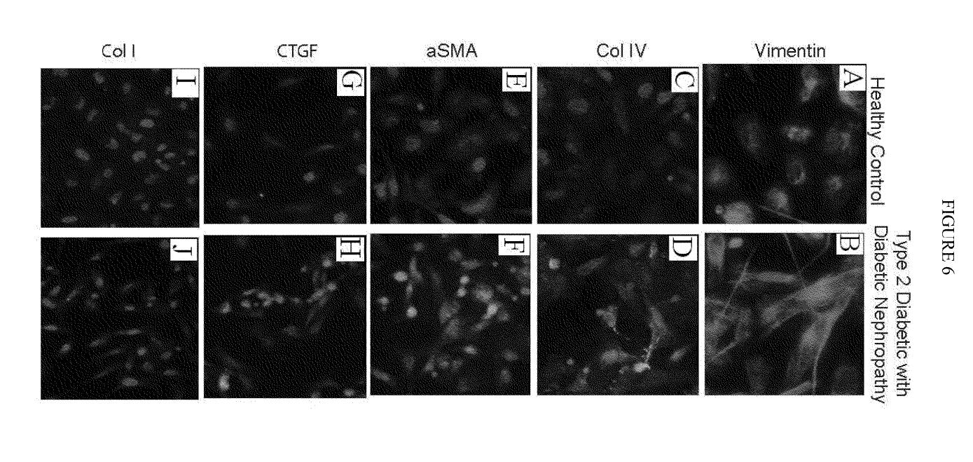

A co-culture fibrosis assay as described in Example 1 and the Materials and Methods was performed using HK-2 cells co-cultured with PBMCs derived from a healthy control, subjects with type 1 diabetes and normoalbuminuria, or subjects with type 1 diabetes and nephropathy. Results are shown in FIG. 5. The results indicate that the PBMC co-culture fibrosis assay is able to distinguish between type 1 diabetic subjects with and without nephropathy based on PBMC-induced fibrotic morphology in HK-2 cells (FIG. 5A-D).

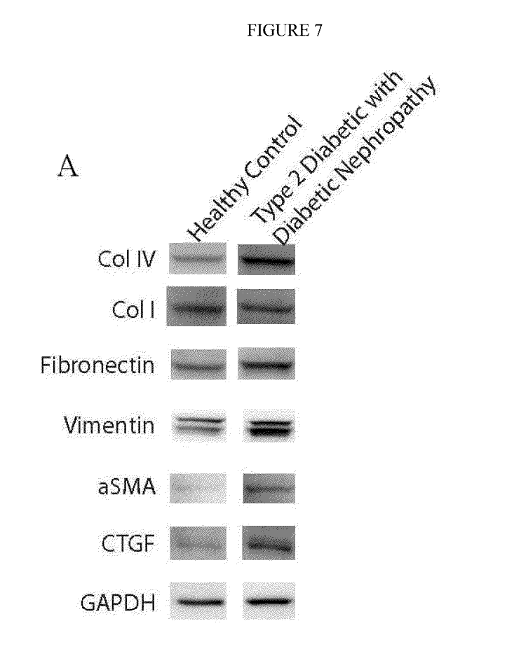

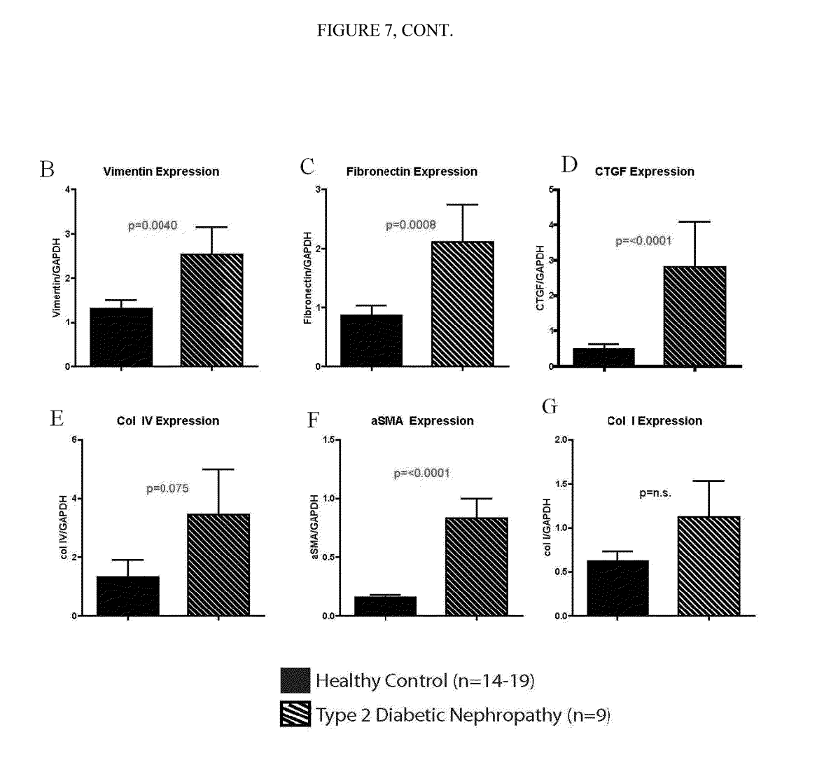

Analysis of extracellular matrix protein production and fibrosis-promoting proteins in the fibrosis assay further distinguished between subjects with type 2 diabetes and diabetic nephropathy, and healthy controls. As shown in FIG. 6, immunocytochemistry performed on the HK-2 cells after co-culture with PBMC from a type 2 diabetic with nephropathy subject displayed an increased production of vimentin, collagen type IV (col IV), aSMA, CTGF, and collagen type I (col I) compared to HK-2 cells after co-culture with PBMC from healthy controls. HK-2 cells after co-culture with PBMC from healthy controls did not display an increase in production of these extracellular matrix proteins. When HK-2 cell lysates made after PBMC co-culture were analyzed by Western Blot to quantify the production of extracellular matrix protein production and fibrosis promoting proteins, co-culture with PBMC from diabetic nephropathy subjects resulted in significantly more production of vimentin (p=0.0040), fibronectin (p=0.0008), CTGF (p<0.0001), and αSMA (p<0.0001) compared to healthy controls (FIG. 7).

### Trans-Well Assay

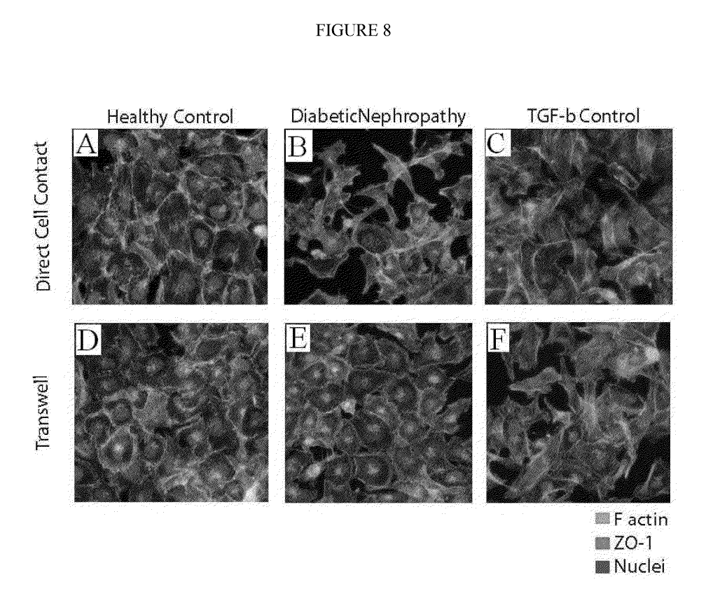

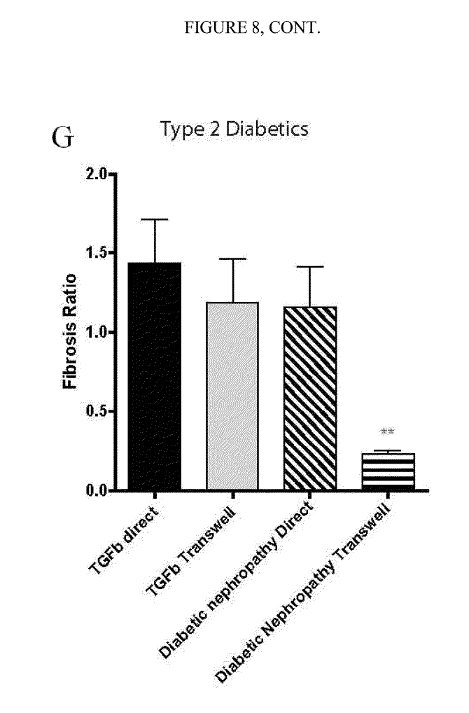

To determine whether direct contact between a subject's PBMCs and renal or epithelial cells is necessary for PBMC-induced fibrotic changes to occur, HK-2 cells were cultured in a trans-well system in which they were separated from the subject's PBMCs by a semipermeable 0.4 μm membrane. By this method, the HK-2 cells are in contacted by PBMC culture supernatant, including compounds secreted by the PBMCs, but not the PBMCs themselves. As shown in FIGS. 8A and 8B, HK-2 cells cultured in the trans-well system did not display morphological changes associated with fibrosis compared to a TGF-β-positive control. These results demonstrate that direct contact between a subject's PBMCs and a cultured cell line is necessary for the PBMCs to induce morphological changes in the cell line that are associated with fibrosis.

This example shows that the methods described herein are useful for detecting the presence diabetes, as well as the presence of or predisposition to insulin resistance, diabetes, and complications of diabetes in a subject. The example further shows that the present methods are useful for detecting the presence of or predisposition to diabetic nephropathy.

### Example 3

### Biomarkers for the Detection of Diabetic Nephropathy and Insulin Resistance by Co-Culture Fibrosis Assay

Biomarkers for detection of the presence of or predisposition to insulin resistance, diabetes and/or complications of diabetes in a subject were identified by comparing the levels of select proteins in culture supernatants and cell lysates from HK-2/PBMC co-culture fibrosis assays performed as described above. PBMCs were isolated from healthy subjects, a pre-diabetic subject, subjects with type 2 diabetes and nephropathy, subjects with long-term diabetes (greater than 10 years) and normoalbuminuria, and subjects with short-term diabetes (less than 5 years) and normoalbuminuria.

Phospho-MAPK Protein Arrays—

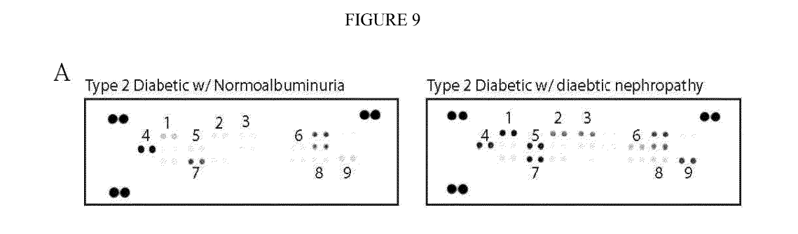

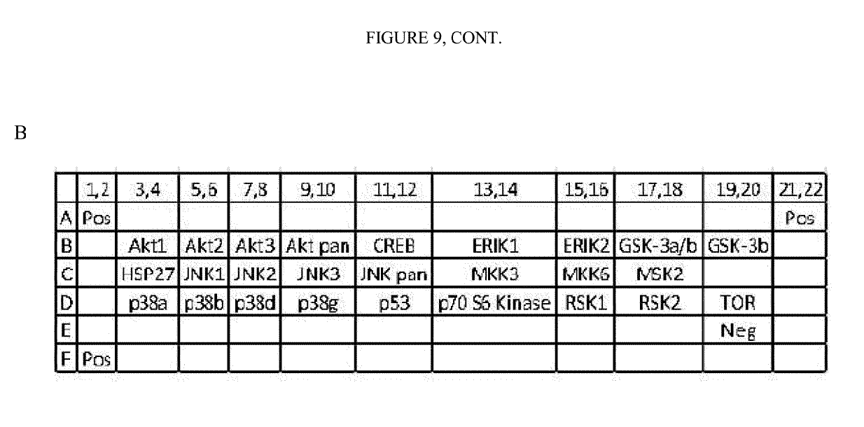

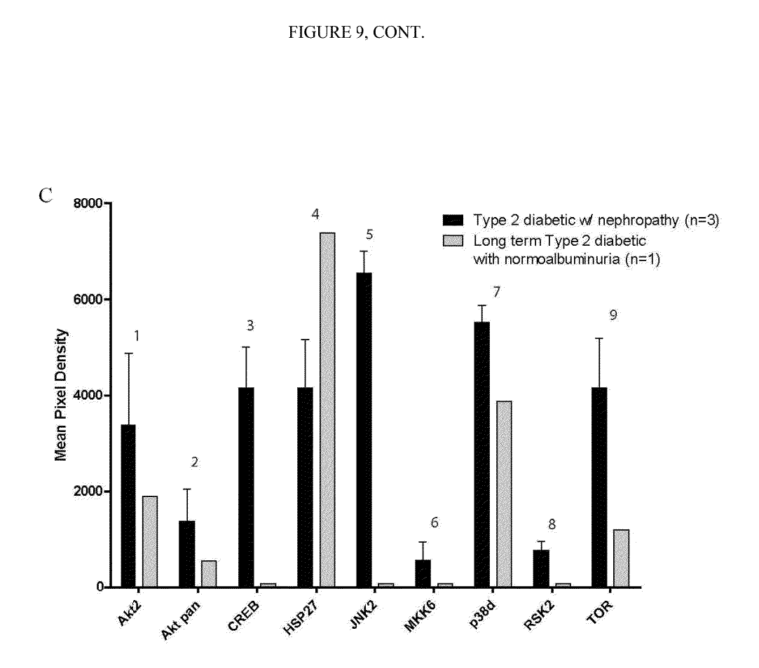

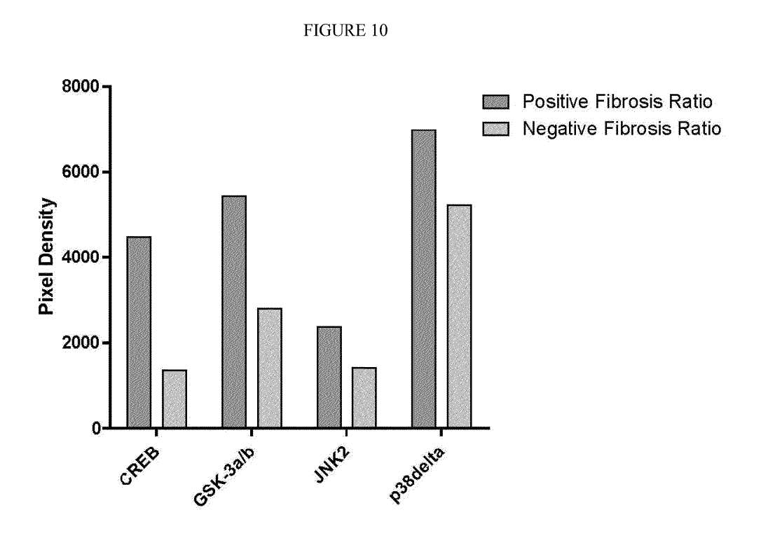

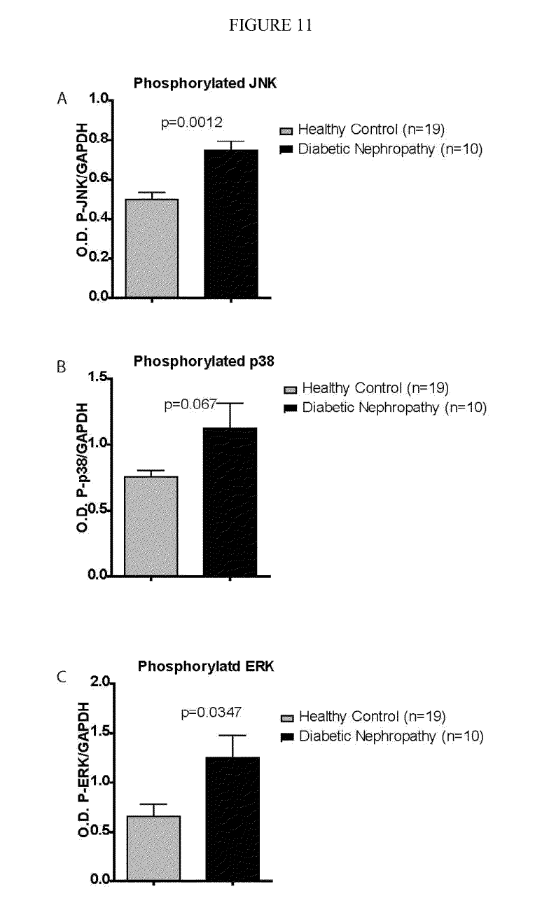

HK-2 cell lysates resulting from co-culture with PBMC were analyzed by protein array for levels of phospho-MAP kinases and kidney biomarkers. PBMCs from type 2 diabetics with nephropathy induced elevation in HK-2 cell levels of phosphorylated Akt2, total Akt, CREB, JNK2, MKK6, p38-delta, RSK2, and TOR, and decreased phosphorylated HSP27 compared to PBMCs from a type 2 diabetic subject with long-term normoalbuminuria and a negative fibrosis ratio (FIG. 9). In addition, PBMCs from type 2 diabetic subjects displayed differing capacities to induce elevations in select biomarkers that correlate with the subject's fibrosis ratio. As shown in FIG. 10, PBMCs from a type 2 diabetic subject with short-term disease duration, normoalbuminuria, and a positive fibrosis ratio induced elevations in levels of phosphorylated CREB, GSK-3α/β, JNK2, and p38-delta, compared to those of a type 2 diabetic subject with short-term disease duration, normoalbuminuria, and a negative fibrosis ratio. This shows that the present methods are useful not only to distinguish between normal and diabetic subjects, but to distinguish between diabetic subjects with differing complications or severity of complications of the disease. These results show that the present methods are useful to predict development of diabetic nephropathy and to detect subclinical nephropathy.

Phospho-MAPK ELISA—

Phospho-MAP kinase ELISAs were performed on HK-2 cell lysates from the PBMC co-culture fibrosis assay for type 2 diabetics with nephropathy and healthy controls. Levels of phosphorylated JNK, phosphorylated p38, and phosphorylated ERK were significantly higher in type 2 diabetics with nephropathy than in controls (FIG. 11A-C). These findings are consistent with the results of protein arrays described above with respect to JNK and p38. However, elevated levels of phosphorylated ERK were not detected by protein array. Without wishing to be bound by theory, this difference may be due to differences in timing of the analysis relative to the period of co-culture. For ELISA sampling, cell lysates were prepared after 24 hours of co-culture, while for protein array analysis, the lysates were prepared following 48 hours of co-culture. The difference may also reflect differential sensitivity of methods.

Soluble Receptors Protein Array—

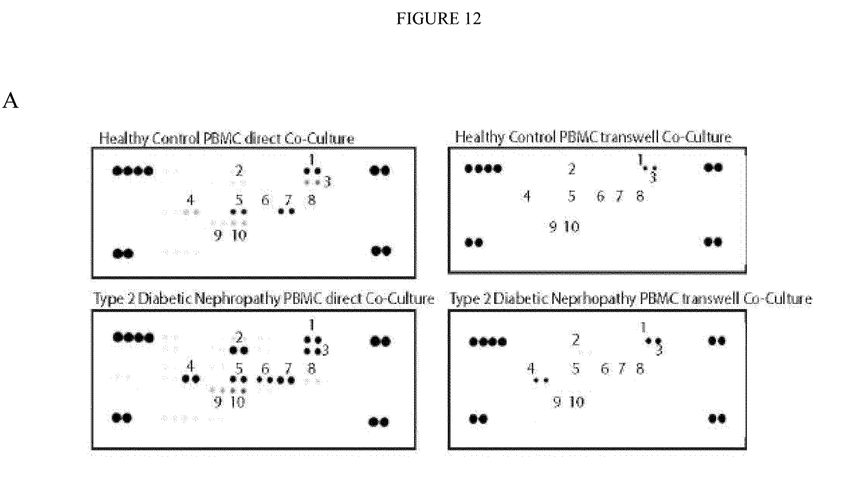

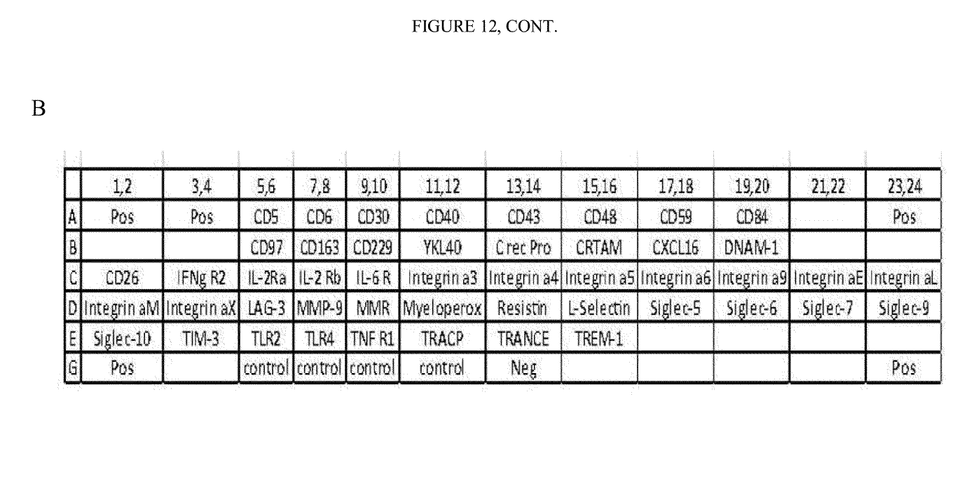

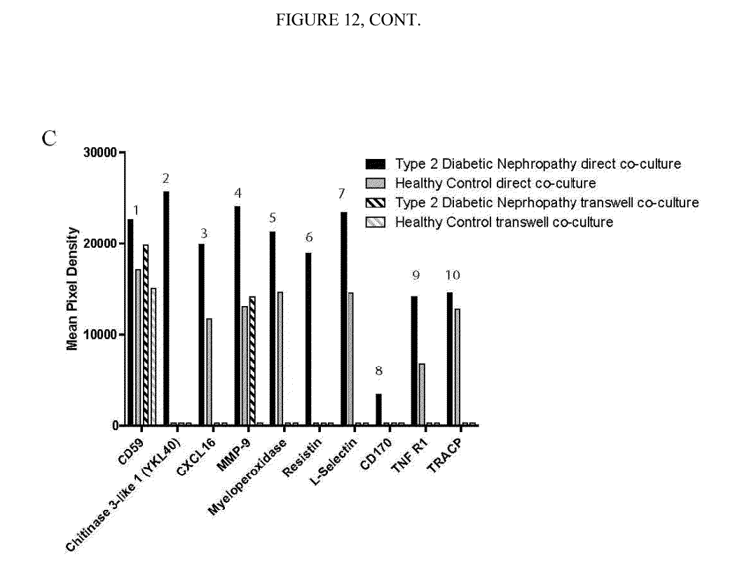

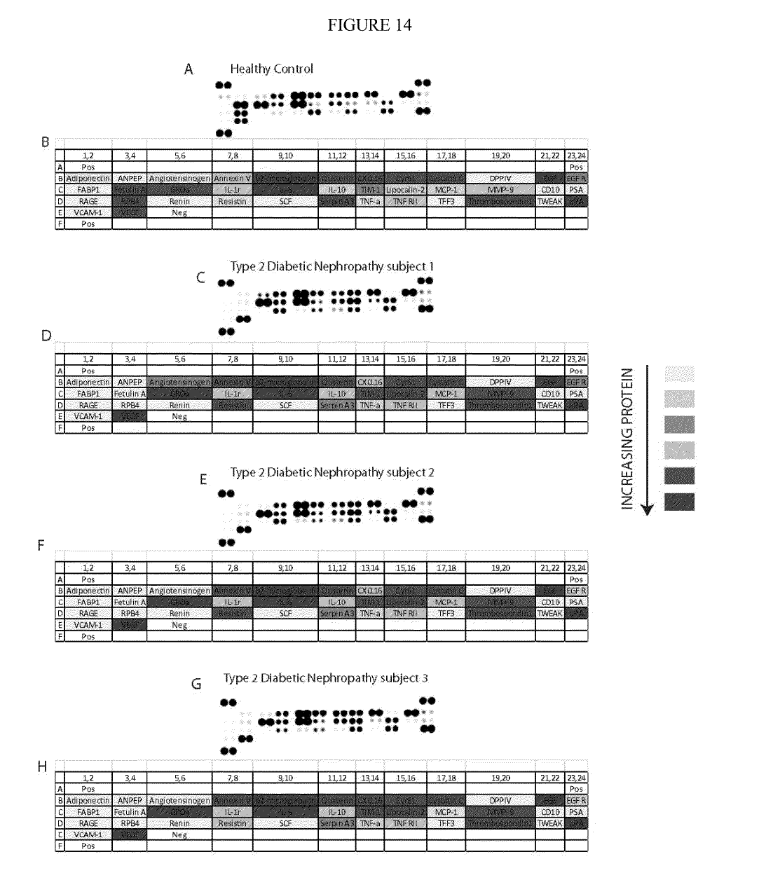

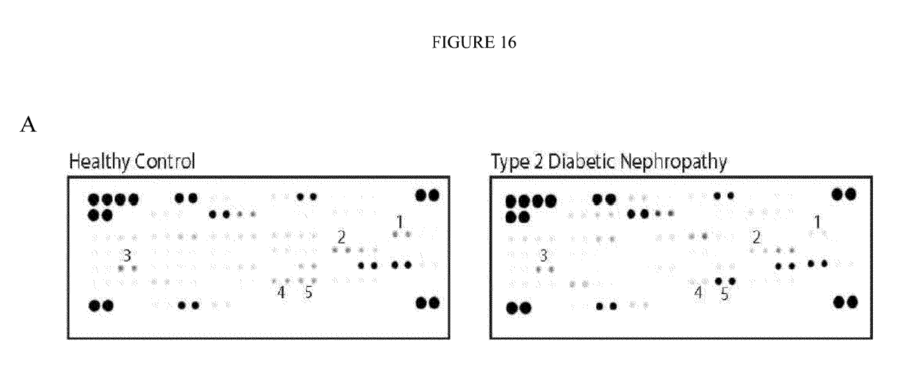

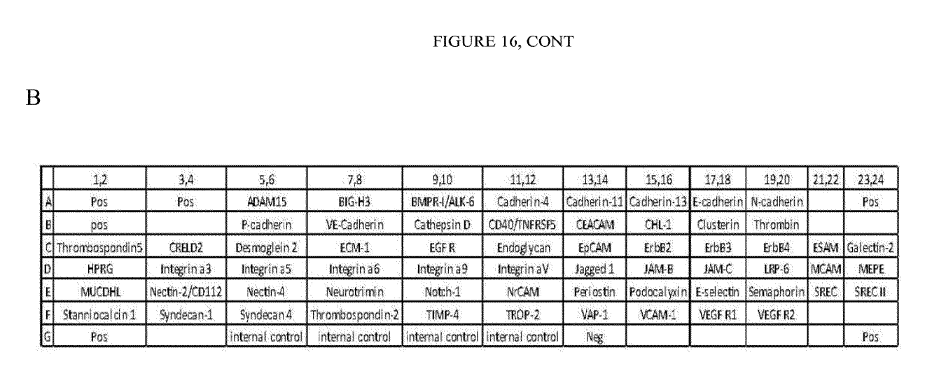

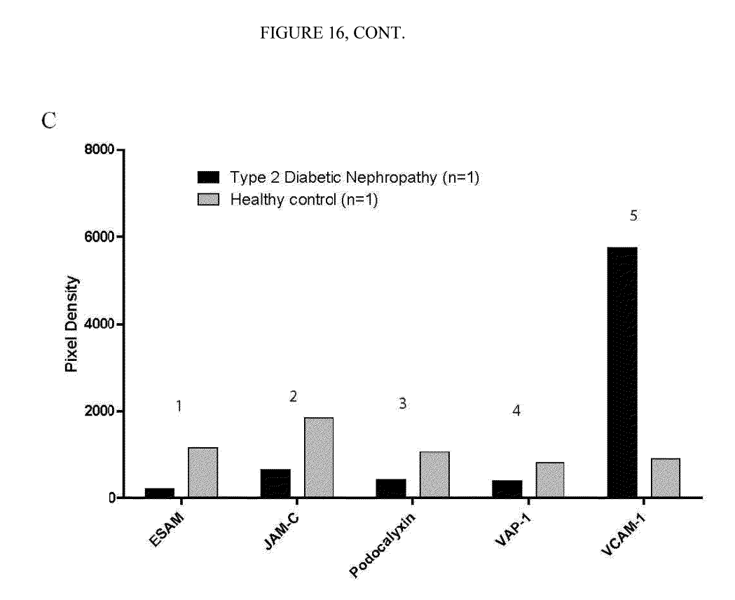

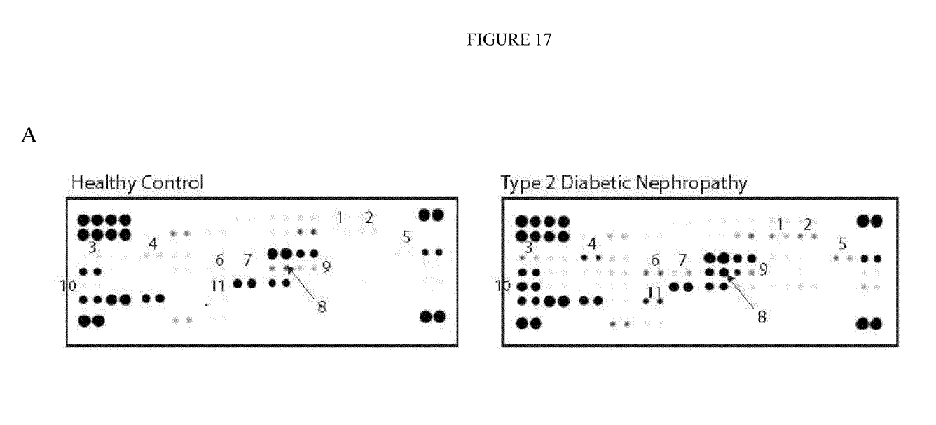

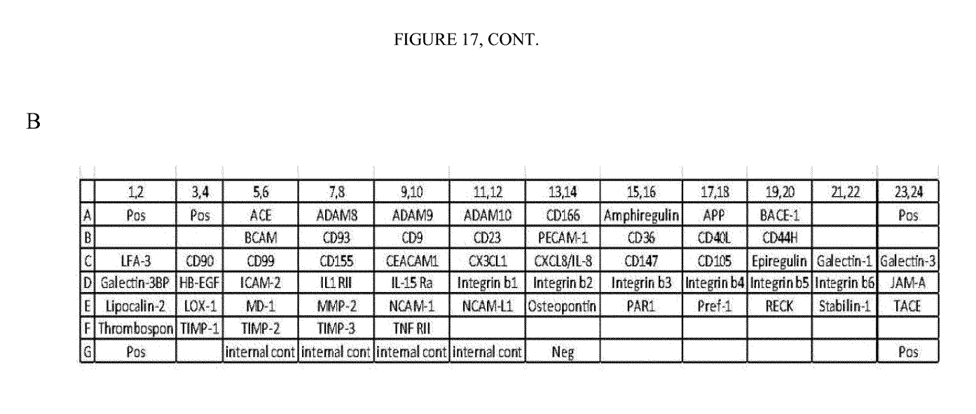

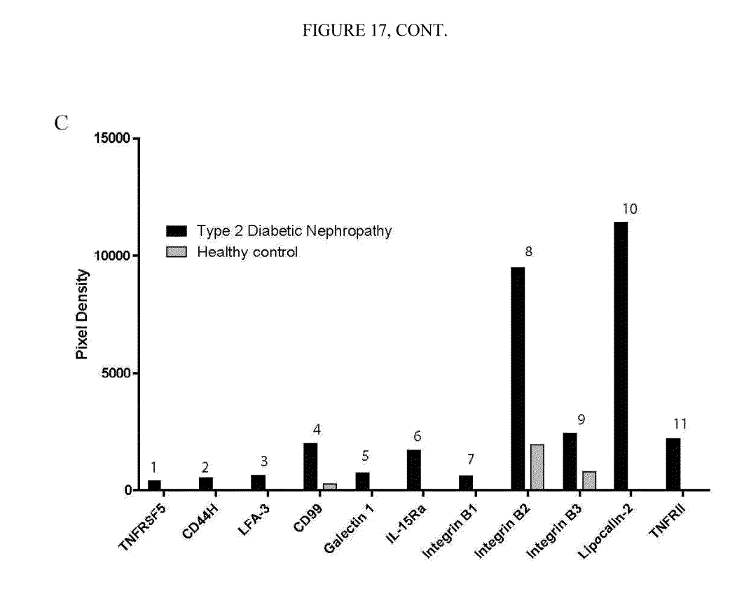

Culture supernatants from co-culture and trans-well fibrosis assays of PBMCs from healthy subjects and type 2 diabetics with nephropathy were analyzed for levels of select hematopoietic soluble receptors (FIG. 12), non-hematopoietic soluble receptors (FIG. 16), and common analyte soluble receptors (FIG. 17). Supernatants from diseased subject samples had measurably higher levels of chitinase, resistin, CD170, VCAM-1, TNFRSF5, CD44H, LFA-3, CD99, galectin 1, IL-15Ra, integrin β1, integrin β2, integrin β3, lipocalin-2, and TNFRII (FIG. 12, 16, 17), and reduced levels of ESAM, JAM-C, podocalyxin, and VAP-1 (FIG. 14) compared to control supernatants.

Kidney Biomarkers Protein Array—

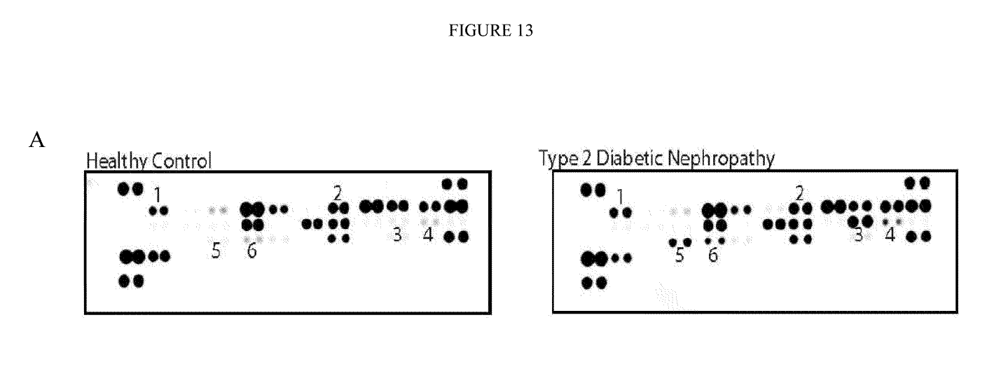

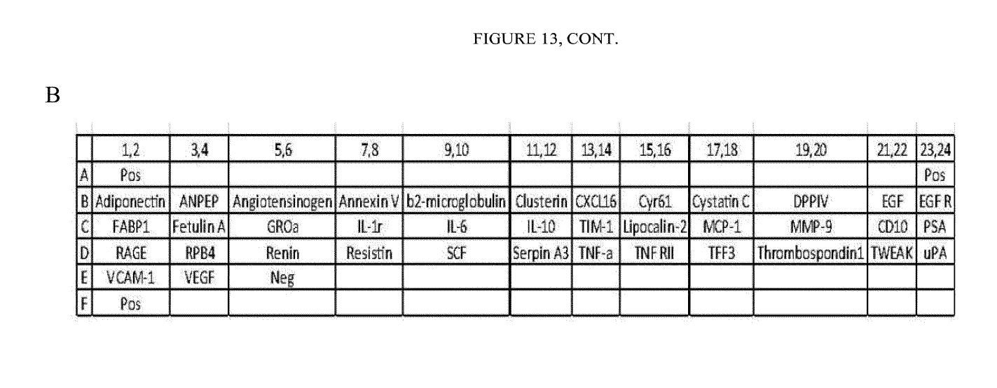

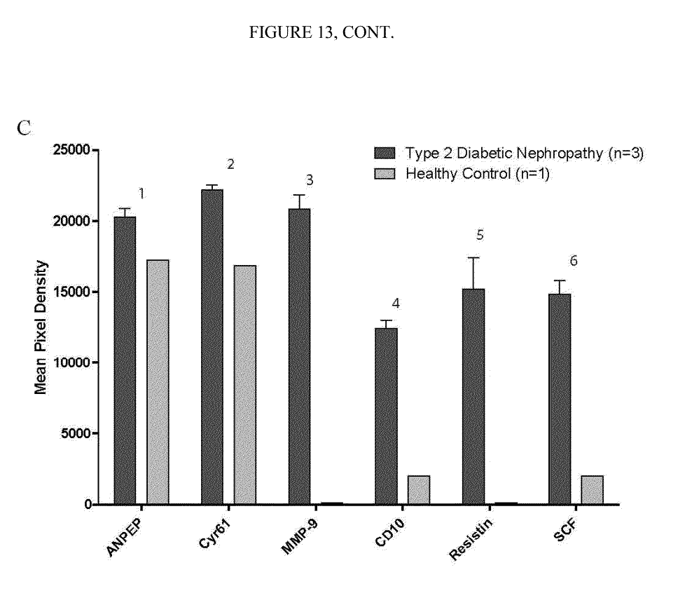

Renal cell lysates from PBMC co-culture assays were analyzed for levels of kidney biomarkers for healthy controls and type 2 diabetics with nephropathy (FIG. 13). Lysates from diseased subject samples had measurably higher levels of MMP-9, CD10, resistin, and SCF compared to a healthy control lysate.

Supernatants from the parallel fibrosis assays were also analyzed. Supernatants from diseased subject samples had measurably higher levels of Annexin V, GRO-α, IL-10, TIM-1, Lipocalin-2, MCP-1, MMP9, Resistin, and VEGF, and measurably lower levels of fetuin A, RPB4, and Serpin A3 (FIG. 14). The results are shown in a heat map format with light green representing background protein levels and dark red representing high levels of protein expression.

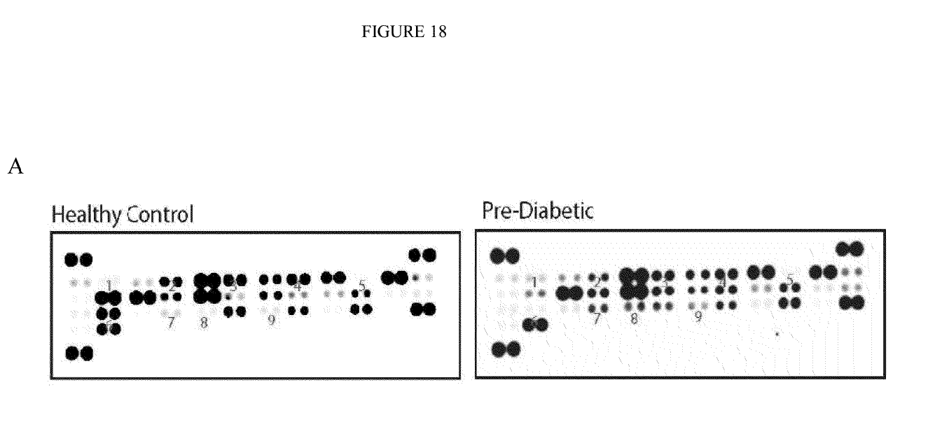

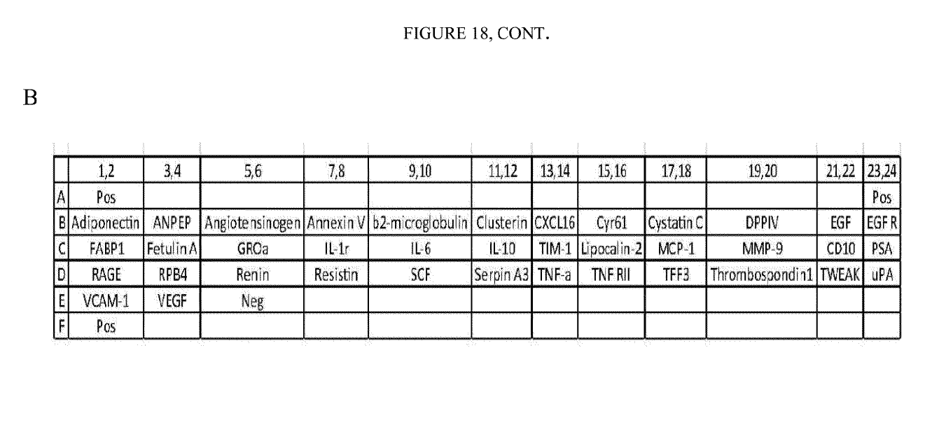

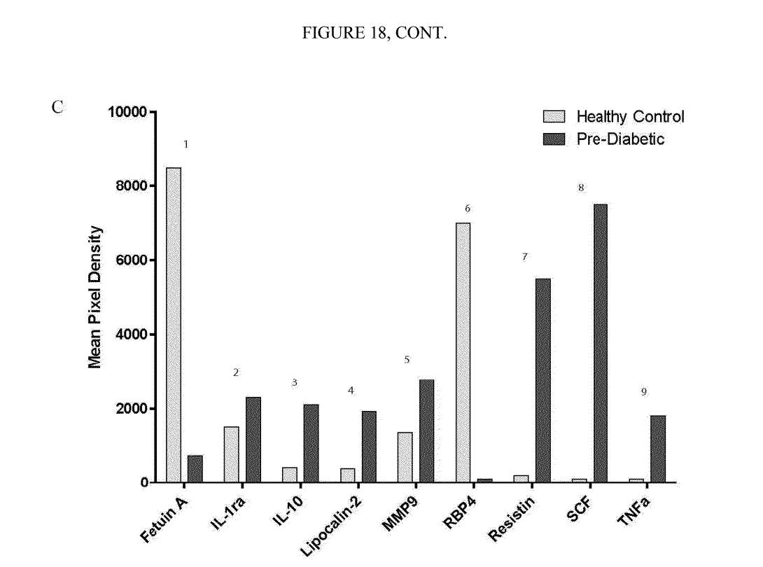

Culture supernatants from co-culture assays of PBMCs from a healthy subject and a pre-diabetic subject were analyzed for levels of kidney biomarkers (FIG. 18). Supernatants from diseased subject samples had measurably higher levels of IL-1Ra, IL-10, Lipocalin-2, MMP-9, Resistin, SCF, and TNFa, and decreased levels of Fetuin A and RBP4 compared to healthy subject samples (FIG. 18).

Kidney Biomarkers in Urine—

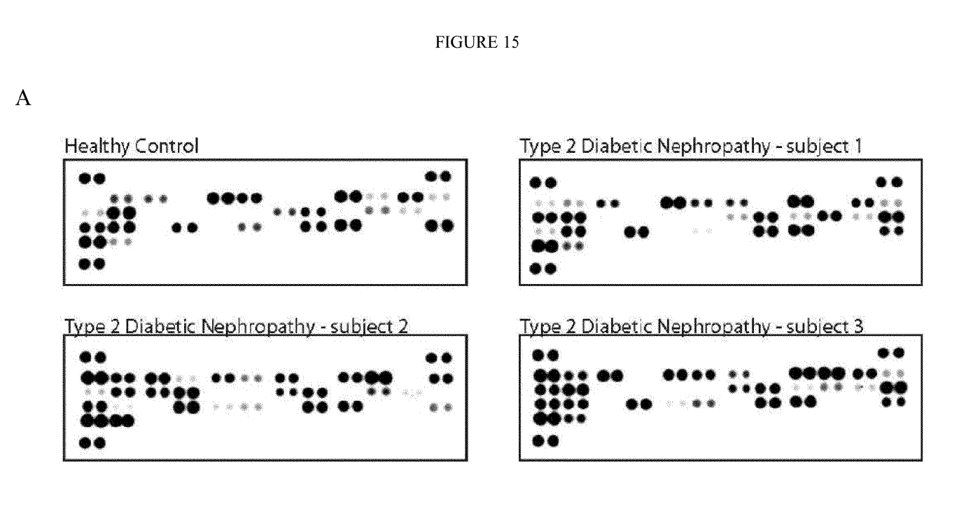

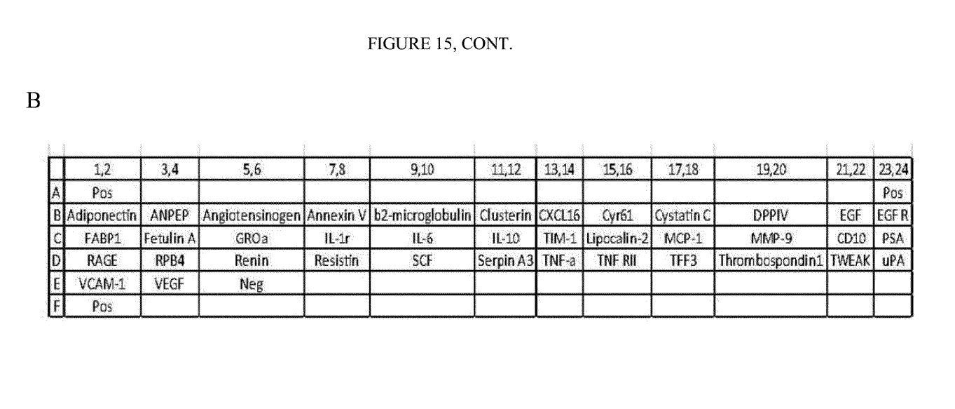

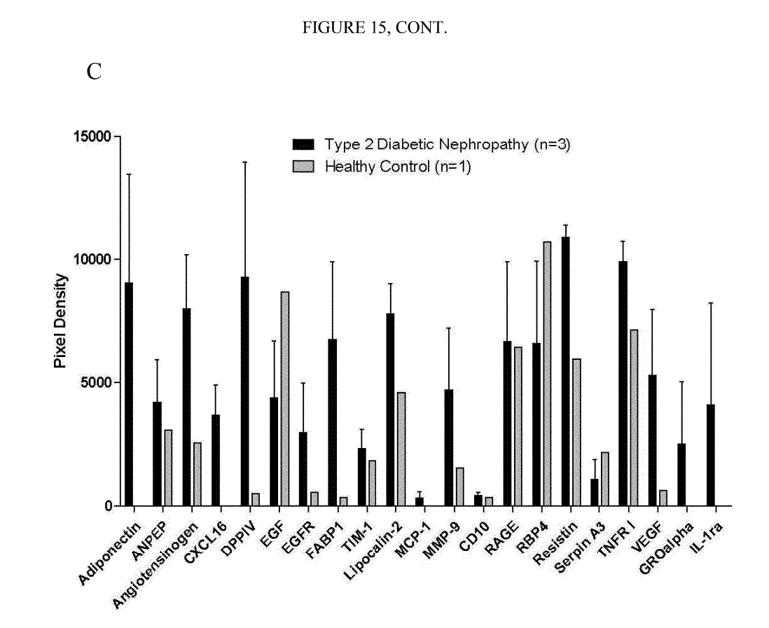

Levels of kidney biomarkers were also measured in urine samples from healthy controls and type 2 diabetics with nephropathy (FIG. 15). The results show that diseased subject urine has measurably higher levels of Adiponectin, ANPEP, Angiotensinogen, DPPIV, EGFR, FABP1, IL-1Ra, CXCL16, TIM-1, Lipocalin-2, MCP-1, MMP-9, CD10, Resistin, TNF R1, VEGF, and GRO-α, and decreased levels of RBP4 and Serpin A3 compared to a healthy control (FIG. 15).

Cytokine Induction—

Select cytokine and chemokine levels were measured in culture supernatants from PBMC co-culture fibrosis assays for healthy subjects and type 2 diabetics with nephropathy. Levels of IL-6, MIP-1α, and MIP-1β were significantly elevated in supernatants from both co-culture and trans-well assays (Table 2, a vs b). By contrast, IL-1β, IL-10, and MCP-1 were considerably higher in supernatants from co-culture assays than from trans-well assays (Table 2, a vs b). This finding shows that while PBMC-induced production of IL-1β, IL-10, and MCP-1 depends on direct contact with a subject's PBMCs, PBMC-induced production of IL-6, MIP-1α, and MIP-1β is contact-independent.

TABLE 2
Cytokine Production in 24 hour co-culture supernatants

| | Direct Cell:Cell co-culture | | Indirect Transwell co-culture | | |
|---|---|---|---|---|---|
| Cytokine | Healthy Normal Controls | T2DM w/ nephropathy | T2DM w/ nephropathy | a vs b | b vs c |
| (pg/ml) | (n = 10) ^{a} | (n = 10) ^{b} | (n = 10) ^{c} | p value | p value |
| IL-1b | 27 ± 35 | 297 ± 204 | 170 ± 108 | <0.0001 | 0.05 |
| TNF-a | 9 ± 8 | 22 ± 18 | 14 ± 11 | 0.03 | 0.067 |
| IL-10 | 18 ± 23 | 162 ± 162 | 14 ± 15 | <0.0001 | 0.03 |
| IL-2 | 25 ± 25 | 45 ± 25 | 48 ± 31 | n.s. | n.s. |
| IL-6 | 2953 ± 1844 | 5947 ± 1233 | 5937 ± 831 | 0.09 | n.s. |
| MCP-1 | 1483 ± 1102 | 11371 ± 7442 | 2434 ± 1125 | <0.0001 | 0.04 |
| MIP-1a | 29 ± 29 | 179 ± 175 | 387 ± 394 | <0.0001 | n.s. |
| MIP-1b | 110 ± 144 | 428 ± 389 | 489 ± 369 | 0.01 | n.s. |
| G-CSF | 1 ± 4 | 7 ± 12 | 3 ± 6 | n.s. | n.s. |
| IL-9 | 6 ± 3 | 6 ± 15 | 4 ± 6 | n.s. | n.s. |
| IFNg | 5 ± 6 | 9 ± 10 | 8 ± 11 | n.s. | n.s. |
| IL-13 | 30 ± 22 | 17 ± 17 | 18 ± 20 | n.s. | n.s. |
| IL-8 | 3151 ± 1069 | 4252 ± 1725 | 4789 ± 1151 | n.s. | n.s. |
| IL-22 | 8 ± 15 | 4 ± 6 | 10 ± 17 | n.s. | n.s. |
| IL-5 | 3 ± 6 | 6 ± 5 | 9 ± 7 | n.s. | n.s. |
| MIG | 2 ± 3 | 1 ± 1 | 3 ± 1 | n.s. | n.s. |
| IL-17 | 2 ± 4 | 2 ± 2 | 1 ± 1 | n.s. | n.s. |
| IL-4 | 0 | 0 | 0 | n.s. | n.s. |
| IL-12p70 | 0 | 0 | 0 | n.s. | n.s. |
| TGFb | 155 ± 152 | 48 ± 61 | 16 ± 39 | n.s. | n.s. |
| IL-18 | 20 ± 63 | 42 ± 84 | 0 | n.s. | n.s. |

These results show that the present methods are useful for detecting the presence of or predisposition to insulin resistance, diabetes and/or complications of diabetes in a subject, including insulin resistance and diabetic nephropathy.

### Example 4

### Analysis of PBMCs Following Co-Culture Fibrosis Assay Distinguishes Type 1 and 2 Diabetics with and without Diabetic Nephropathy from Healthy Controls

This example will demonstrate that PBMCs co-cultured with HK-2 cells display changes in physiology and morphology associated with fibrosis, and that analysis of the PBMCs may be used to distinguish between diabetic subjects with nephropathy and healthy subjects.

PBMCs from healthy subjects or subjects with type 2 diabetes and nephropathy, microalbuminuria, or normoalbuminuria are co-cultured with HK-2 cells in the co-culture fibrosis assay described in Example 1. Following co-culture with HK-2 cells, PBMCs are isolated and analyzed for morphological and physiological changes according to the present methods.

Morphological and Physiological Changes—

PBMCs from healthy subjects are predicted to display little or no alteration in morphology compared to untreated cells. By contrast, PBMCs from diabetic subjects with nephropathy are predicted to display alterations in morphology and physiology characteristic of fibrosis, including increased spindle formation, re-distribution of F-actin and ZO-1, and re-organization of F-actin into stress fibers.

Fibrosis Ratios—

Fibrosis ratios will be calculated for PBMCs from (1) healthy controls; (2) short-term (less than 5 years) type 2 diabetic subjects with normoalbuminuria; (3) long-term (greater than 10 years) type 2 diabetic subjects with microalbuminuria; and (4) type 2 diabetics with nephropathy. It is predicted that PBMCs from diseased subjects will have fibrosis ratios significantly higher than PBMCs from healthy subjects.

The results are predicted to show that PBMC fibrosis ratios are useful for distinguishing between healthy subjects and subjects with diabetic nephropathy. The results are further predicted to show that fibrosis ratios are useful to distinguish between type 2 diabetics with microalbuminuria, and early indicator of diabetic nephropathy, from healthy subjects, and to distinguish between short-term type 2 diabetics with normoalbuminuria, who may or may not develop diabetic neuropathy, and long-term type 2 diabetics with normoalbuminuria, who generally do not develop diabetic neuropathy.

Computer-Assisted Analysis—

The fibrosis ratios described above will be reproduced using the CellProfiler morphological analysis software as described in the Materials and Methods. After staining, cells are analyzed using the CellProfiler, which quantifies phenotypical changes in the samples. It is predicted that CellProfiler analysis will re-iterate the results described above relating to changes in PBMC morphology following co-culture with HK-2 cells.

Organ-Resident Cell Type Specificity—

It is predicted that PBMCs from diabetics with nephropathy will display morphological and physiological changes when co-cultured with renal cells, but not with cell lines derived from other organs or tissues. These results will demonstrate that PBMCs have a specific response to cell types derived from organs that are compromised in the individual from which they are isolated, as opposed to a general capacity to undergo alterations associated with in response to ay cell-cell contact generally.

These results will show that the present methods are useful for detecting the presence of or predisposition to insulin resistance, diabetes and/or complications of diabetes in a subject, including insulin resistance and diabetic nephropathy.

### Example 5

### PBMC Biomarkers for the Detection of Diabetic Nephropathy and Insulin Resistance by Co-Culture Fibrosis Assay

This example will demonstrate that PBMCs used for fibrosis assays of the present technology display changes in levels of biomarkers following co-culture with renal cells.

PBMCs are isolated from healthy subjects, subjects with type 2 diabetes and nephropathy, subjects with long-term diabetes (greater than 10 years) and normoalbuminuria, or subjects with short-term diabetes (less than 5 years) and normoalbuminuria. Following co-culture with HK-2 cells, the PBMCs are isolated and analyzed for biomarker expression according to the methods described above.

Phospho-MAPK Protein Arrays—

PBMC cell lysates are analyzed by protein array for levels of phospho-MAP kinases and kidney biomarkers. PBMCs from type 2 diabetics with nephropathy are predicted to display elevated levels of phosphorylated Akt2, total Akt, CREB, JNK2, MKK6, p38-delta, RSK2, and TOR, and decreased phosphorylated HSP27 compared to PBMCs from a type 2 diabetic subject with long-term normoalbuminuria and a negative fibrosis ratio. PBMCs from a type 2 diabetic subject with short-term disease duration, normoalbuminuria, and a positive fibrosis ratio are predicted to display elevated levels of phosphorylated CREB, GSK-3α/β, JNK2, and p38-delta, compared to those of a type 2 diabetic subject with short-term disease duration, normoalbuminuria, and a negative fibrosis ratio.

These results will show that the present methods are useful to distinguish between normal and diabetic subjects, and to distinguish between diabetic subjects with differing complications or severity of complications of the disease. These results will further show that PBMCs cultured according to the present methods are useful for predicting development of diabetic nephropathy and to detect subclinical nephropathy.

Phospho-MAPK ELISA—

Lysates of PBMCs cultured as described above are analyzed for phospho-MAP kinase levels by ELISA. It is predicted that levels of phosphorylated JNK, phosphorylated p38, and phosphorylated ERK will be significantly higher in PBMCs from type 2 diabetics with nephropathy than in healthy controls. These findings will be consistent with the results of protein arrays described above with respect to JNK and p38.

Soluble Receptors Protein Array—

Lysates of PBMCs cultured as described above are analyzed for levels of select hematopoietic soluble receptors, non-hematopoietic soluble receptors, and common analyte soluble receptors. Supernatants from diseased subject samples are predicted to have measurably higher levels of chitinase, resistin, CD170, VCAM-1, TNFRSF5, CD44H, LFA-3, CD99, galectin 1, IL-15Ra, integrin β1, integrin β2, integrin β3, lipocalin-2, and TNFRII, and reduced levels of ESAM, JAM-C, podocalyxin, and VAP-1 compared to control supernatants.

Kidney Biomarkers Protein Array—

Lysates of PBMCs from healthy controls and type 2 diabetics with nephropathy cultured as described above are analyzed for levels of kidney biomarkers for healthy controls and type 2 diabetics with nephropathy. Lysates from diseased subject samples are predicted to have measurably higher levels of MMP-9, CD 10, resistin, and SCF compared to healthy control lysates.

These results will show that the present methods are useful for detecting the presence of or predisposition to insulin resistance, diabetes and/or complications of diabetes in a subject, including insulin resistance and diabetic nephropathy.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

1. A method for identifying a subject as having a predisposition to diabetic nephropathy, comprising:
(a) co-culturing a biological sample from the subject in vitro with one or more renal cell lines;
(b) maintaining the co-culture for a sufficient time for the biological sample to induce physiological changes in the renal cells; and
(c) detecting the physiological changes in the renal cells;
wherein the subject is asymptomatic for diabetic nephropathy.

2. The method of claim 1, wherein the biological sample comprises peripheral blood mononuclear cells (PBMCs) or urine.

3. The method of claim 1, wherein the subject is an individual diagnosed as having, suspected of having, or predisposed to having one or more diseases or conditions selected from the group consisting of type 1 diabetes, type 2 diabetes, insulin resistance, normalbuminuria, and microalbuminuria.

4-5. (canceled)

6. The method of claim 1, wherein the physiological changes comprise changes in cell or cell culture morphology.

7. The method of claim 6, wherein the changes in cell or cell culture morphology comprise changes associated with fibrosis.

8. The method of claim 7, wherein the changes in cell or cell culture morphology associated with fibrosis comprise one or more of changes selected from the group consisting of spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, and enhanced individual cell migration.

9. The method of claim 6, wherein the changes in cell or cell culture morphology comprise one or more changes selected from the group consisting of cell area, compactness, eccentricity, extent, solidity, angle between neighbors, radial distribution, angular second movement, contrast, difference entropy, difference variance, entropy, inverse difference moment, sum average, sum variance, and variance.

10. (canceled)

11. The method of claim 1, wherein the physiological changes comprise changes in protein level comprising an increase in one or more proteins selected from the group consisting of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII, IL-1β, IL-10, MIP-1α, MIP-1β, phospho-CREB, DPPIV, EGF, EGFR, TIM-1, TNF-α, VEGF, annexin V, angiotensin, CXCL16, MCP-1, GRO-α, and IL-1Ra.

12. The method of claim 1, wherein the physiological changes comprise changes in protein level comprising a decrease in one or more proteins selected from the group consisting of phospho-HSP27, JAM-C, podocalyxin, and VAP-1.

13. The method of claim 1, wherein the detecting comprises one or more methods selected from the group consisting of microscopy, immunostaining, ELISA, protein arrays, western blotting, and flow cytometry.

14. A method for identifying a subject as diabetic, comprising:
(a) co-culturing a biological sample from the subject in vitro with one or more renal cell lines;
(b) maintaining the co-culture for a sufficient time for the biological sample to induce physiological changes in the renal cells; and
(c) detecting the physiological changes in the renal cells;
wherein the subject is asymptomatic for diabetic nephropathy.

15. The method of claim 14, wherein the biological sample comprises peripheral blood mononuclear cells (PBMCs) or urine.

16. The method of claim 14, wherein the subject is an individual suspected of having or predisposed to having one or more diseases or conditions selected from the group consisting of type 1 diabetes, type 2 diabetes, insulin resistance, normalbuminuria, microalbuminuria, and macroalbuminuria.

17-18. (canceled)

19. The method of claim 14, wherein the physiological changes comprise changes in cell or cell culture morphology.

20. The method of claim 19, wherein the changes in cell or cell culture morphology comprise changes associated with fibrosis.

21. The method of claim 20, wherein the changes in cell or cell culture morphology associated with fibrosis comprise one or more of changes selected from the group consisting of spindle formation, cell elongation, increased cell contractility/mobility, increased proliferation, increased apoptosis, increased necrosis, decreased viability, reduced cell-cell contact, increased filapodial stress fibers, cytoskeletal reorganization, decreased tight intercellular junctions, formation of focal adhesions, and enhanced individual cell migration.

22. The method of claim 19, wherein the changes in cell or cell culture morphology comprise one or more changes selected from the group consisting of cell area, compactness, eccentricity, extent, solidity, angle between neighbors, radial distribution, angular second movement, contrast, difference entropy, difference variance, entropy, inverse difference moment, sum average, sum variance, and variance.

23. (canceled)

24. The method of claim 14, wherein the physiological changes comprise changes in protein level comprising an increase in one or more proteins selected from the group consisting of vimentin, fibronectin, connective tissue growth factor (CTGF), alpha smooth muscle actin (αSMA), collagen IV, collagen I, phospho-Akt 2, total phospho-Akt, phospho-JNK2, phopho-MKK6, phospho-p38δ, phosphor-RSK2, target of rapamycin, GSK-3α/β, phospho-ERK, CD59, chitinase 3-like 1, MMP-9 myeloperoxidase, resistin, L-selectin, CD170, TNF-R1, TRACAP, ANPEP, Cyr61, CD10, SCF, VCAM-1, TNFRSF5, CD44H, LFA-3, CD99, galectin 1, IL15Ra, integrin β1, integrin β2, integrin β2, lipocalin-2, TNF-RII, IL-1β, IL10, MIP-1α, MIP-1β, phospho-CREB, DPPIV, EGF, EGFR, TIM-1, TNF-α, VEGF, annexin V, angiotensin, CXCL16, MCP-1, GRO-α, and IL-1Ra.

25. The method of claim 14, wherein the physiological changes comprise changes in protein level comprising a decrease in one or more proteins selected from the group consisting of phospho-HSP27, JAM-C, podocalyxin, and VAP-1.

26. The method of claim 14, wherein the detecting comprises one or more methods selected from the group consisting of microscopy, immunostaining, ELISA, protein arrays, western blotting, and flow cytometry.

27-47. (canceled)
